(19) United States
(12) Patent Application Publication
(10) Pub. No.: US 2014/0113978 A1
(43) Pub. Date: Apr. 24, 2014

(54) MULTIFOCAL HEPATOCELLULAR CARCINOMA MICRORNA EXPRESSION PATTERNS AND USES THEREOF

(71) Applicants: Christopher Barry, Rochester, NY (US); Tony Godfrey, Rochester, NY (US); Anthony Almudevar, Pittsford, NY (US)

(72) Inventors: Christopher Barry, Rochester, NY (US); Tony Godfrey, Rochester, NY (US); Anthony Almudevar, Pittsford, NY (US)

(73) Assignee: UNIVERSITY OF ROCHESTER, Rochester, NY (US)

(21) Appl. No.: 14/115,258

(22) PCT Filed: May 1, 2012
(86) PCT No.: PCT/US2012/036002

§ 371 Date: Jan. 7, 2014

Related U.S. Application Data
(60) Provisional application No. 61/481,207, filed on May 1, 2011.

(51) Int. Cl. C12Q 1/68; G06F 19/24

(52) U.S. Cl. CPC C12Q 1/6886; G06F 19/24
USPC 514/789; 435/6.11; 506/9; 702/19

(57) ABSTRACT

The present invention is directed to methods and products for defining a biomarker of disease phenotype. The present invention further relates to methods and kits for determining a subject's risk of developing recurrent hepatocellular carcinoma based on a defined microRNA biomarker that reliably distinguishes hepatocellular carcinoma disease recurrence from non-recurrence. The invention also relates to methods of treating a patient having heptocellular carcinoma based on their risk of developing hepatocellular carcinoma disease recurrence.

This application claims the benefit of U.S. Provisional Patent Application Ser. No. 61/481,207, filed May 1, 2011, which is hereby incorporated by reference in its entirety.

### FIELD OF THE INVENTION

The present invention is directed to methods and kits for determining a subject's risk of developing recurrent hepatocellular carcinoma, and methods of treating a patient based on this determined risk. The present invention also relates to methods and products for defining a biomarker of disease phenotype.

### BACKGROUND OF THE INVENTION

Hepatocellular carcinoma (HCC) is one of the most common cancers worldwide (Bruix & Sherman, “Management of Hepatocellular Carcinoma,” Hepatology 42:1208-1236 (2005); Yang & Roberts, “Epidemiology and Management of Hepatocellular Carcinoma,” Infect. Dis. Clin. North Am. 24:899-919 (2010)) and is a major cause of cancer mortalities particularly in Africa and Asia (Bosch et al., “Primary Liver Cancer: Worldwide Incidence and Trends,” Gastroenterology 127(Suppl 1):S5-S16 (2004)). The incidence of HCC is increasing in western countries due to the hepatitis C virus epidemic (El-Serag et al., “The Continuing Increase in the Incidence of Hepatocellular Carcinoma in the United States: An Update,” Ann. Intern. Med. 139:817-823 (2003)) and, more recently, the obesity epidemic leading to nonalcoholic steatohepatitis (Ekstedt et al., “Long-Term Follow-Up of Patients with NAFLD and Elevated Liver Enzymes,” Hepatology 44:865-873 (2006)). Curative treatment is currently limited to surgical resection and liver transplantation, but resection results in recurrence rates of greater than 70% within 5 years (Imamura et al., “Risk Factors Contributing to Early and Late Phase Intrahepatic Recurrence of Hepatocellular Carcinoma After Hepatectomy,” J. Hepatol. 38:200-207 (2003)) (6) and most patients (80%) present with extensive disease that is not amenable to surgery (Tang Z. Y., “Hepatocellular Carcinoma—Cause, Treatment and Metastasis,” World J. Gastroenterol. 7:445-454 (2001)). Transplanting within the Milan criteria (1 tumor≦5 cm, 2-3 tumors≦3 cm, and no evidence of intrahepatic vascular invasion or extrahepatic spread) is the accepted standard of care as 5-year survival rates of 80% or greater and 5-year recurrence rates of less than 15% can be achieved (Mazzaferro et al., “Liver Transplantation for the Treatment of Small Hepatocellular Carcinomas in Patients with Cirrhosis,” N. Engl. J. Med. 334:693-699 (1996)). However, these criteria are based primarily on radiographic characteristics and do not assess individual tumor biology and risk of recurrence or overall survival. Clearly, there are some patients outside of Milan criteria who have a low incidence of recurrence and there are those within Milan who do suffer from recurrent HCC after transplant. A biomarker indicative of a tumor's propensity for recurrence would be of great value in optimizing overall patient outcomes.

There is increasing evidence, primarily from global genomic studies (Budhu et al., “The Molecular Signature of Metastases of Human Hepatocellular Carcinoma,” Oncology 69(Suppl 1):23-27 (2005); Bernards and Weinberg, “A Progression Puzzle,” Nature 418:823 (2002)), that metastatic potential is inherent in the primary tumor from an early stage and that this information can be used to predict long-term outcomes. Several groups have begun to study this in HCC using microarray technology to define messenger RNA (mRNA) and microRNA (miRNA) expression profiles that correlate with survival, recurrence and metastatic disease in the hopes of describing clinically useful biologic metrics to guide patient selection and appropriate therapeutic interventions (Ye et al., “Predicting Hepatitis B Virus-Positive Metastatic Hepatocellular Carcinomas Using Gene Expression Profiling and Supervised Machine Learning,” Nat. Med. 9:416 123 (2003); Lee et al., “Classification and Prediction of Survival in Hepatocellular Carcinoma by Gene Expression Profiling,” Hepatology 40:667-676 (2004); Iizuka et al., “Predicting Individual Outcomes in Hepatocellular Carcinoma,” Lancet 364:1837-1839 (2004); Hoshida et al., “Gene Expression in Fixed Tissues and Outcome in Hepatocellular Carcinoma,” N. Engl. J. Med. 359:1995-2004 (2008); Budhu et al., “Identification of Metastasis-Related MicroRNAs in Hepatocellular Carcinoma,” Hepatology 47:897-907 (2008); Sato et al., “MicroRNA Profile Predicts Recurrence After Resection in Patients With Hepatocellular Carcinoma Within the Milan Criteria,” PLoS One 6:e16435 (2011); Toffanin et al., “MicroRNA-Based Classification of Hepatocellular Carcinoma and Oncogenic Role of miR-517a,” Gastroenterology 140:161.8-1.628 (2011)). However, HCC is frequently associated with multifocal ahepatic disease and this causes problems for defining such gene expression signatures. Intrahepatic metastasis arises from local dissemination of the primary tumor and can account for up to 757© of multifocal lesions whereas de novo lesions (multiple primary tumors) arise as a result of the diseased liver milieu that is predisposed to oncogenesis (Tarao et al., “Role of Increased DNA Synthesis Activity of Hepatocytes in Multicentric Hepatocarcinogenesis in Residual Liver of Hepatectomized Cirrhotic Patients With Hepatocellular Carcinoma,” Jpn. J. Cancer Res. 85:1040-1044 (1994); Brady et al., “Frequency and Predictors of De Novo Hepatocellular Carcinoma in Patients Awaiting Orthotopic Liver Transplantation During the Model for End-Stage Liver Disease Era,” Liver Transpl. 14:228-234 (2008); Chen et al., “Clonal Origin of Recurrent Hepatocellular Carcinomas,” Gastroenterology 96:527-529 (1989); Hsu et al., “Clonality and Clonal Evolution of Hepatocellular Carcinoma With Multiple Nodules,” Hepatology 13:923-928 (1991); Paradis et al., “Clonal Analysis of Macronodules in Cirrhosis,” Hepatology 28:953-958 (1998); Cheung et al., “Identify Metastasis-Associated Genes in Hepatocellular Carcinoma Through Clonality Delineation for Multinodular Tumor,” Cancer Res. 62:4711 1721 (2002)). Assessment of mRNA or miRNA expression profiles from single tumors fails to address the problem of multifocal tumors and their different expression signatures.

Thus, in a patient with multifocal HCC, there remains a need for a biomarker that takes into account the possibility that the tumors are not clonally related and have different genetic profiles and associated metastatic potential.

The present invention is directed to overcoming these and other limitations in the art.

### SUMMARY OF THE INVENTION

A first aspect of the present invention is directed to a method of determining a subject's risk of developing recurrent hepatocellular carcinoma. This method involves contacting an isolated hepatocellular carcinoma sample from the subject with reagents suitable for detecting expression levels of two or more microRNAs in the sample and measuring the expression levels of the two or more microRNAs in the sample based on said contacting. The method further involves calculating a risk score of hepatocellular carcinoma disease recurrence for the subject based on the measured microRNA expression levels in the isolated sample and comparing the calculated risk score for the subject to a reference threshold score of hepatocellular carcinoma disease recurrence to determine the subject's risk of developing recurrent hepatocellular carcinoma.

Another aspect of the present invention is directed to a method of treating a subject having hepatocellular carcinoma. This method involves calculating the subject's risk score of hepatocellular carcinoma disease recurrence based on measured expression levels of two or more microRNAs in one or more isolated hepatocellular carcinoma samples from the subject and comparing the calculated risk score for the subject to a reference threshold score of hepatocellular carcinoma disease recurrence. A suitable therapy for the subject is administered based on the calculated risk score.

Another aspect of the present invention is directed to a kit comprising a collection of oligonucleotides, said collection consisting essentially of two or more oligonucleotides that hybridize under stringent conditions to two or more microRNAs, respectively, wherein the two more microRNAs are selected from the group of hsa-miR-501, hsa-miR-1180, hsa-miR-365, hsa-miR-1273, hsa-miR-377, hsa-let-7d, hsa-miR-576, hsa-miR-454, hsa-miR-18a, hsa-miR-15a, hsa-miR-548c, hsa-miR-20a, hsa-miR-610, miR-146b, hsa-miR-137, hsa-miR-1293, hsa-miR-139, hsa-miR26a, hsa-miR-122, hsa-miR-192, hsa-miR-888, hsa-miR-497, hsa-miR-592, hsa-miR-545, hsa-miR-5|3u, hsa-miR-136, hsa-miR-1226, hsa-miR-651, hsa-miR-542, hsa-miR-491, hsa-miR-937, hsa-miR-424, hsa-miR-630, hsa-miR-33b, hsa-miR-615, hsa-mir-152, hsa-miR-455, hsa-miR-23b, hsa-miR-671, hsa-miR-30c-2, hsa-miR-193b, hsa-miR-1260, hsa-miR-505, hsa-miR-181c, hsa-miR-99a, hsa-miR-885, hsa-miR-145, hsa-miR-194, hsa-miR-125b-2, hsa-miR-182.

Another aspect of the present invention is directed to a method of defining a biomarker reference threshold score that correlates with a disease phenotype. This method involves obtaining one or more disease samples from each individual in a cohort of patients having different disease phenotypes. The one or more obtained disease samples are contacted with reagents suitable for detecting expression levels of two or more candidate molecular biomarkers, and the minimum and maximum expression levels of the candidate molecular biomarkers within the one or more disease samples from each individual are measured based on said contacting. This method further involves selecting the minimum and/or maximum expression levels of the candidate molecular biomarkers that significantly correlate with a disease phenotype of the patient cohort and generating a standardized expression value for each of the selected minimum and maximum expression levels. The method further involves constructing a biomarker reference score for each individual in the cohort by summing the standardized expression values of said candidate molecular biomarkers whose inclusion in the sum maximizes the correlation between the biomarker reference score and the disease phenotype, and summarizing biomarker reference score distribution across the cohort to define a biomarker reference threshold score.

Another aspect of the present invention is directed to a method of defining a biomarker reference threshold score that correlates with a disease phenotype. This method involves, obtaining from at least one or more sources, by a statistical computing device, minimum and maximum expression levels of candidate molecular biomarkers in one or more disease samples from each individual in a cohort of patients having different disease phenotypes. This method further involves selecting, by the statistical computing device, the minimum and/or maximum expression levels of the candidate molecular biomarkers that significantly correlate with a disease phenotype of the patient cohort, and generating, by the statistical computing devise, a standardized expression value for each of the selected minimum and maximum expression levels of the molecular biomarkers. The method further involves constructing, by the statistical computing device, a biomarker reference score for each individual in the cohort by summing the standardized expression values of said candidate molecular biomarkers whose inclusion in the sum maximizes the correlation between the biomarker reference score and the disease phenotype, and summarizing, by the statistical computing device, biomarker reference score distribution across the cohort to define a biomarker reference threshold score.

Another aspect of the present invention is directed to non-transitory computer readable medium having stored thereon instructions for defining a biomarker reference threshold score that correlates with a disease phenotype comprising machine executable code which when executed by at least one processor, causes the processor to perform steps that include, obtaining from one or more sources, minimum and maximum expression levels of candidate molecular biomarkers within one or more disease samples from each individual in a cohort of patients having different disease phenotypes and selecting the minimum and/or maximum expression levels of the candidate molecular biomarkers that significantly correlate with a disease phenotype in the patient cohort. The steps further include generating a standardized expression value for each of the selected minimum and maximum expression levels of the molecular biomarkers: constructing a biomarker reference score for each individual in the cohort by summing the standardized expression values of said candidate molecular biomarkers whose inclusion in the sum maximizes the correlation between the biomarker reference score and the disease phenotype, and summarizing biomarker reference score distribution across the cohort to define a biomarker reference threshold score

Another aspect of the present invention is directed to a computing device for defining a biomarker reference threshold score that correlates with a disease phenotype. This device includes one or more processors and a memory device coupled to the one or more processors, wherein the one or more processors is configured to execute programmed instructions stored in the memory device. These instructions include obtaining minimum and maximum expression levels of candidate molecular biomarkers in one or more disease samples from individuals in a cohort of patients having different disease phenotypes and selecting the minimum and/or maximum expression levels of the candidate molecular biomarkers that significantly correlate with a disease phenotype in the patient cohort. The instructions further include generating a standardized expression value for each of the selected minimum and maximum expression levels of the molecular biomarkers, constructing a biomarker reference score for each individual in the cohort by summing the standardized expression values of said candidate molecular biomarkers whose inclusion in the sum maximizes the correlation between the biomarker reference score and the disease phenotype, and summarizing biomarker reference score distribution across the cohort to define a biomarker reference threshold score.

HCC is frequently associated with multifocal intrahepatic disease and this causes problems for defining such gene expression signatures. Intrahepatic metastasis arises from local dissemination of the primary tumor and can account for up to 75% of multifocal lesions whereas de novo lesions (multiple primary tumors) arise as a result of the diseased liver milieu that is predisposed to oncogenesis (Tarao et al., “Role of Increased DNA Synthesis Activity of Hepatocytes in Multicentric Hepatocarcinogenesis in Residual Liver of Hepatectomized Cirrhotic Patients With Hepatocellular Carcinoma,” Jpn. J. Cancer Res. 85:1040-1044 (1994); Brady et al., “Frequency and Predictors of De Novo Hepatocellular Carcinoma in Patients Awaiting Orthotopic Liver Transplantation During the Model for End-Stage Liver Disease Era,” Liver Transpl. 14:228-234 (2008); Chen et al., “Clonal Origin of Recurrent Hepatocellular Carcinomas,” Gastroenterology 96:527-529 (1989); Hsu et al., “Clonality and Clonal Evolution of Hepatocellular Carcinoma With Multiple Nodules,” Hepatology 13:923-928 (1991); Paradis et al., “Clonal Analysis of Macronodules in Cirrhosis,” Hepatology 28:953-958 (1998); Cheung et al., “Identify Metastasis-Associated Genes in Hepatocellular Carcinoma Through Clonality Delineation for Multinodular Tumor,” Cancer Res. 62:4711-4721 (2002), which are hereby incorporated by reference in their entirety). Thus, in a patient with multifocal HCC, any improved biomarker must take into account the possibility that the tumors are not clonally related and, thus, will have different genetic profiles and associated metastatic potential. To address this, the methods of the present invention (referred to as MIN-MAX method) account for multiple expression patterns of the same miRNA in patients with multifocal disease by assessing both the minimum and maximum miRNA expression levels across all tumors in a single patient. As described herein, applicants define a miRNA biomarker that reliably distinguishes patients with and without HCC recurrence after liver transplant. The biomarker described herein can be used alone or in conjunction with the current Milan criteria to improve selection decisions in liver transplant candidates with HCC. Furthermore, such a metric of HCC tumor biology could also be used to direct other therapeutic interventions such as surgical resection, ablative therapy, chemotherapy and radiation. Based on the improved predictive value of the present invention, the invention can be applied to biomarker identification for other diseases.

### BRIEF DESCRIPTION OF THE DRAWINGS

FIG. 1 is an exemplary environment comprising a statistical computing device for defining a biomarker reference score that correlates with a particular disease phenotype.

FIG. 2 is a flowchart depicting an exemplary method of defining a biomarker reference score that correlates with a particular disease phenotype.

FIG. 3 is a flowchart depicting an exemplary method of determining a subject's risk of developing recurrent HCC using an HCC biomarker developed in accordance with the present invention.

FIG. 4 is a representative electrophoresis gel showing consistently high yields of miRNA (pink arrows) from FFPE tumor blocks using the Roche High Pure miRNA kit.

FIG. 5 is a representative scatter plot of microarray hybridization results comparing miRNA purified from freshly frozen cells (x-axis) and identical cells that were first fixed with formalin and paraffin embedded (FFPE, y-axis) prior to miRNA purification. Cells used were from the OE-19 esophageal cancer cell line, harvested during exponential growth phase. Purified miRNA was labeled and hybridized to Affymetrix GeneChip miRNA 1.0 microarrays as described in the Examples.

FIGS. 6A-6C show unsupervised hierarchical clustering dendograms of all 88 samples assayed (FIG. 6A), 64 samples after MIN/MAX reduction (FIG. 6B) and MIN/MAX reduced probes with a false discovery rate (FDR)<0.2 (FIG. 6C). Recurrent samples are shown as solid squares.

FIG. 7 depicts a principal component analysis of recurrence (red points) and nonrecurrence (black points) samples. The principal components (PC) of a multivariate data set are orthogonal components of a linear transformation of the data. The first PC is the one of maximum variance, and the remaining are ranked in decreasing order by variance (the maximum possible under the orthogonality constraint). As shown in analysis of FIG. 7, the first principal component (x-axis) accounts for most of the variability in the data and separates recurrence from nonrecurrence similarly to the hierarchical clustering analysis (FIGS. 6A-6C).

FIG. 8 shows the 67 miRNA probes used to distinguish patients with and without HCC recurrence after liver transplant. Hierarchical clustering heatmap of individual patients (x-axis) versus miRNAs (y-axis). The miRNAs are ordered by difference in average expression between recurrence and nonrecurrence (most upregulated in recurrence at the top). The horizontal black line divides those upregulated in recurrence from those downregulated in recurrence. Color bar in upper right corner denotes relative expression levels (blue=upregulated, red downregulated). Individual patient recurrence status is shown at the top (black=recurrent HCC within 3 years of liver transplant, yellow=no recurrence). MIN and MAX refer to minimum and maximum miRNA expression levels, respectively, as defined hereinafter.

FIGS. 9A-9C are Kaplan-Meier curves of recurrence-free survival as delineated by the miRNA biomarker (i.e., the biomarker reference threshold score) in the entire cohort (FIG. 9A), patients outside Milan criteria at time of transplant (FIG. 9B) and patients within Milan criteria (FIG. 9C).

FIG. 10 is a box plot showing the sensitivity of mean miRNA expression probe aggregation or summary versus min/max aggregation or summary.

FIG. 11 shows receiver operator characteristic curves of miRNA biomarker performance relative to Milan (red dots) in distinguishing patients with and without recurrent disease. Area under curve (AUC) values are shown for Min-Max reduced miRNAs in addition to Milan criteria (top left), Min-Max reduction alone (top right), mean expression reduced miRNAs in addition to Milan (bottom left) and mean reduction alone (bottom right). The Min-Max method, alone or in combination with Milan criteria, outperformed prior biomarkers.

FIG. 12 is a graph of the R-squared values (y-axis) versus number of probes (x-axis) illustrating performance of Min-Max versus mean miRNA expression probe reduction with and without the addition of Milan criteria.

FIG. 13 shows the cross validation procedures as a function of maximum number of probe features (M). AUC statistics for Min-Max with Milan (-) Min-Max alone (---), mean of multifocal expression values with Milan () and mean alone (--).

FIG. 14 depicts plots showing expression of three representative miRNAs comprising the biomarker: 497, 194, and 125b-2-star with relative expression on the y-axis and individual patients on the x-axis. Boxes show the spread of expression values for that miRNA in patients with multifocal disease.

FIGS. 15A-15D are graphs displaying recurrence risk scores for tissue-based (FIGS. 15A-B) and subject-based biomarkers (FIG. 15C-15D; subject-based biomarker employs Min/Max aggregation). Risk scores are displayed by subject. For the tissue-based biomarker, multiple risk scores for a single subject are represented by boxplots. In each case, the dashed line represents an estimate of the risk score with 90% specificity.

FIGS. 16A-16E present a table of recurrent hepatocellular carcinoma biomarker microRNAs and their sequences (SEQ ID NOs: 1-65).

### DETAILED DESCRIPTION OF THE INVENTION

The present invention relates to methods for the identification of biomarkers of disease or disease phenotypes, as well as methods for the implementation of the biomarker to assess the disease phenotype of a patient, or identify a treatment regimen suitable for the patient. Systems and devices for the implementation of these methods are also disclosed.

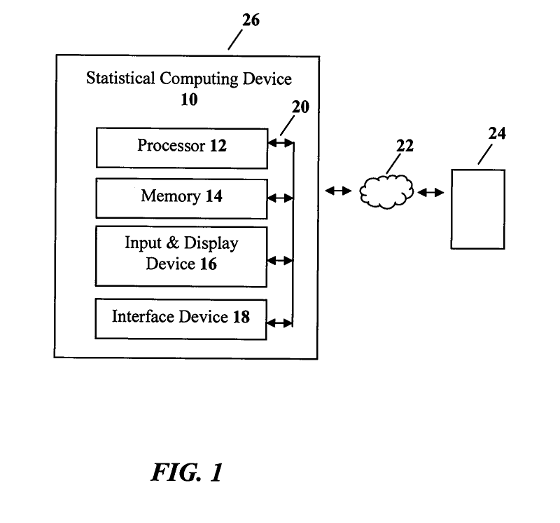

An exemplary environment 26 with a statistical computing device 10 that defines a biomarker reference score that correlates with a particular disease phenotype is illustrated in FIG. 1. The environment 26 includes statistical computing device 10, communication network 22 and patient database server 24, although the environment can include other types and numbers of devices, components, elements and communication networks in other topologies and deployments. This technology/process provides a number of advantages including providing more effective methods, non-transitory computer readable medium and devices for generating and defining a biomarker that reliably distinguishes disease phenotypes.

The statistical computing device 10 assists with defining a biomarker reference threshold score that correlates with a disease phenotype, although the statistical computing device 10 may perform other types and numbers of functions. The statistical computing device 10 includes at least one processor 12, memory 14, input and display devices 16, and interface device 18 which are coupled together by a bus 20 or other link, although the statistical computing device 10 may comprise other types and numbers of elements in other configurations.

Processor(s) 12 may execute one or more computer-executable instructions stored in the memory 14 for the methods illustrated and described with reference to the examples herein, although the processor(s) can execute other types and numbers of instructions and perform other types and numbers of operations. The processor(s) 12 may comprise one or more central processing units (“CPUs”) or general purpose processors with one or more processing cores, such as AMD® processor(s), although other types of processor(s) could be used (e.g., Intel®).

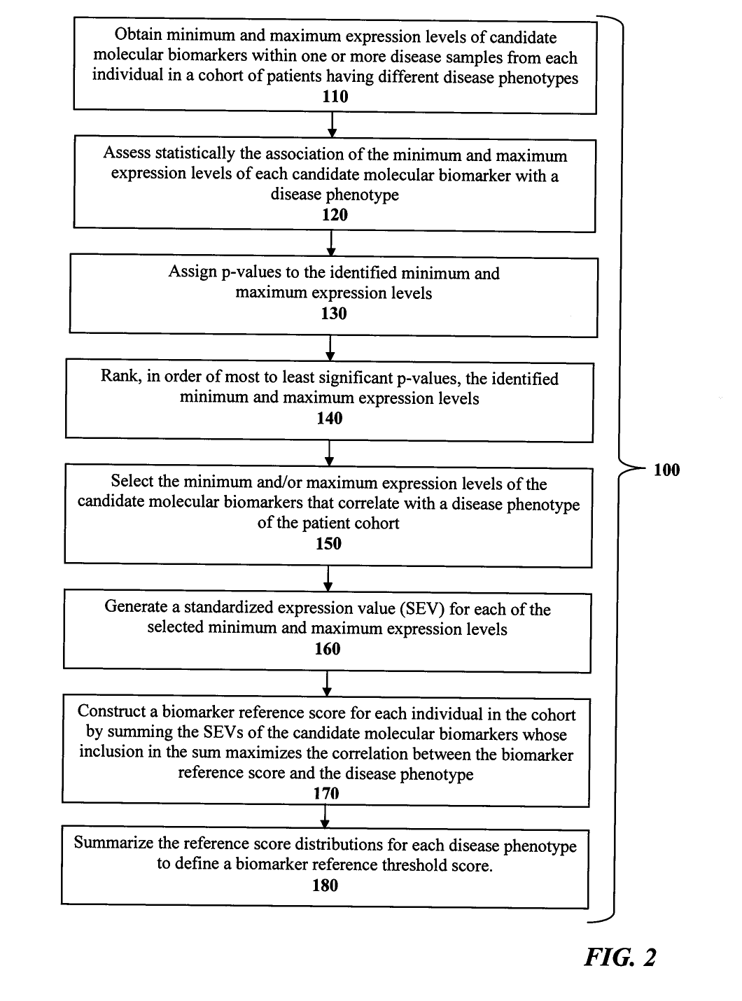

Memory 14 may comprise one or more tangible storage media, such as RAM, ROM, flash memory, CD-ROM, floppy disk, hard disk drive(s), solid state memory, DVD, or any other memory storage types or devices, including combinations thereof, which are known to those of ordinary skill in the art. Memory 14 may store one or more non-transitory computer-readable instructions of this technology as illustrated and described with reference to the examples herein that may be executed by the one or more processor(s) 12. The flow chart 100 shown in FIG. 2 is representative of example steps or actions of this technology that may be embodied or expressed as one or more non-transitory computer or machine readable instructions stored in memory 14 that may be executed by the processor(s) 12.

Input and display devices 16 enable a user, such as an administrator, to interact with statistical computing device 10, such as to input and/or view data and/or to configure, program, and/or operate it by way of example only. Input devices may include a keyboard and/or a computer mouse and display devices may include a computer monitor, although other types and numbers of input devices and display devices could be used.

The interface device 18 in the statistical computing device 10 is used to operatively couple and communicate between the statistical computing device 10 and the patient database server 24 which are coupled together by a communication network. The communication network 22 can be a local area network (LAN) and/or a wide area network (WAN), although other types and numbers of communication networks or systems with other types and numbers of connections and configurations to other devices and elements can also be used. By way of example only, the local area networks (LAN) and the wide area network (WAN) can use TCP/IP over Ethernet and industry-standard protocols, including NFS, CIFS, SOAP, XML, LDAP, and SNMP, although other types and numbers of communication networks, can be used. In this example, the bus 20 is a hyper-transport bus, although other bus types and links may be used, such as PCI.

The patient database server 24 processes requests received from the statistical computing device 10 via communication network 22 according to the HTTP-based application, RFC protocol, the CIFS or NFS protocol, or other application protocols. A series of applications may run on the patient database server 24 that allow the transmission of data, such as patient specific biomarker data, e.g., mRNA, miRNA, or other expression data derived from a patient sample, requested by the statistical computing device 10. The patient database server 24 may provide data or receive data in response to requests directed toward the respective applications on the patient database server 24 from the statistical computing device 10. It is to be understood that the patient database server 24 may be hardware or software or may represent a system with multiple servers 16, which may include internal or external networks. In this example the patient database server 24 may be any version of Microsoft® IIS servers or Apache® servers, although other types of servers may be used.

Furthermore, each of the systems of the examples may be conveniently implemented using one or more general purpose computer systems, microprocessors, digital signal processors, and micro-controllers, programmed according to the teachings of the examples, as described and illustrated herein, and as will be appreciated by those of ordinary skill in the art.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including, by way of example only, teletraffic in any suitable form (e.g., voice and modem), wireless traffic media, wireless traffic networks, cellular traffic networks, G3 traffic networks, Public Switched Telephone Network (PSTNs), Packet Data Networks (PDNs), the Internet, intranets, and combinations thereof.

The examples may also be embodied as a non-transitory computer readable medium having instructions stored thereon for one or more aspects of the technology as described and illustrated by way of the examples herein, which when executed by a processor (or configurable hardware), cause the processor to carry out the steps necessary to implement the methods of the examples, as described and illustrated herein.

The method of defining a biomarker reference threshold score that correlates with a disease phenotype in accordance with the exemplary embodiments will now be described with reference the flow chart 100 of FIG. 2.

In step 110 the statistical computing device 10 obtains from one or more sources, minimum and maximum expression levels of candidate molecular biomarkers in one or more disease samples from each individual in a cohort of patients having different disease phenotypes. As used herein, “different disease phenotypes” encompasses any clinically distinct phenotype, stage, or status of a particular disease. By way of example only, different disease phenotypes may include, but are not limited to, recurrent disease and non-recurrent disease (e.g., recurrent hepatocellular carcinoma and non-recurrent hepatocellular carcinoma), metastatic and non-metastatic cancer or disease (including solid tumor cancers and non-solid tumor cancers), relapse disease and non-relapse disease. Other disease states in which biomarker discovery would be informative, include, for example, Non-Alcoholic Fatty Liver Disease and Non-Alcoholic Steato-Hepatitis.

In one embodiment of this aspect of the present invention, the statistical computing device 10 obtains the minimum and maximum expression levels of candidate molecular biomarkers in step 110 from a patient database server 24. The patient database server 24 stores all past and presently available expression data for individuals of a cohort of patients, as well as, expression data obtained in the future from the same cohort or different cohorts of patients.

Alternatively, the statistical computing device 10 obtains the minimum and maximum expression levels of candidate molecular biomarkers from step 110 by measuring the levels directly from a sample. In accordance with this embodiment, the values of the sample are stored in the patient database server 24, although they can also be stored within the memory 14. The method of measurement depends on the candidate molecular biomarker that is being measured as described in more detail below. Accordingly, suitable candidate molecular biomarkers include, without limitation, mRNA expression levels, microRNA, snRNA, snoRNA, and scaRNA expression levels (collectively referred to hereinafter as “microRNA” or “miRNA”), protein expression levels including protein isoform expression levels, and metabolite concentrations.

When the candidate molecular biomarkers comprise mRNA expression levels and/or microRNAs expression levels, suitable methods for measuring the expression of these biomarkers include, without limitation, hybridization-based assays, amplification-based assays, and next generation sequencing.

In a hybridization assay, expression is measured based on the hybridization of one or more oligonucleotide probes to at least a portion of a nucleotide sequence comprising a microRNA or mRNA nucleic acid molecule whose expression is to be measured. The oligonucleotide probe or probes comprise a nucleotide sequence that is complementary to at least a region of a microRNA, mRNA, or corresponding cDNA.

As used herein, the term “hybridization” refers to the complementary base-pairing interaction of one nucleic acid with another nucleic acid that results in formation of a duplex, triplex, or other higher-ordered structure. Typically, the primary interaction is base specific, A/T and G/C, by Watson/Crick and Hoogsteen-type hydrogen bonding. Base-stacking and hydrophobic interactions can also contribute to duplex stability. Conditions for hybridizing detector probes and primers to complementary and substantially complementary target sequences are well known in the art (see e.g., NUCLEIC ACID HYBRIDIZATION, A PRACTICAL APPROACH, B. Hames and S. Higgins, eds., IRL Press, Washington, D.C. (1985), which is hereby incorporated by reference in its entirety). In general, hybridization is influenced by, among other things, the length of the polynucleotides and the complementary, the pH, the temperature, the presence of mono- and divalent cations, the proportion of G and C nucleotides in the hybridizing region, the viscosity of the medium, and the presence of denaturants. Such variables influence the time required for hybridization. Thus, the preferred hybridization conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and single stranded nucleic acid probe. Thus, complementarity herein is meant that the probes or primers are sufficiently complementary to the target sequence to hybridize under the selected reaction conditions to achieve selective detection and measurement.

High-throughput hybridization assays carried out using microarray platforms are particularly suitable for carrying out the methods of the present invention. In an array based method a series of oligonucleotide probes are affixed to a solid support. The cDNA or RNA biomarkers in a sample from the subject are labeled and the sample is contacted with the array containing the oligonucleotide probes. Hybridization of nucleic acid molecules from the sample to their complementary oligonucleotide probes on the array surface is detected using a suitable array reading device and then measured. The array reading device can be any of a variety of known devices (e.g., Affymetrix GeneChip Scanner 3000 7G System and IIlumina BeadArray and BeadXpress Readers and iScan instruments) that export the measured array data to the statistical computing device 10 via communication network 22 or other suitable input or, alternatively, the statistical computing device of the present invention can directly carry ou reading of the array. Examples of direct hybridization array platforms include, without limitation, the Affymetrix GeneChip arrays and Illumina Bead Array.

In an amplification based assay, oligonucleotide primers and, optionally, probes, are utilized to amplify and measure the expression level of molecular biomarker, i.e., cDNA or RNA, within a patient sample. In certain embodiments, the assay is a high-throughput and multiplex assay. Commonly used amplification assays are the quantitative polymerase chain reaction (qPCR) assay and quantitative real-time qRT-PCR assays. In some embodiments of the present invention, particularly where the biomarker is microRNA or mRNA, the qPCR assay is preceded by a reverse transcription step. Numerous qPCR and qRT-PCR amplification and detection chemistries are known in the art and are suitable for use in the methods of the present invention. By way of example only, these include, without limitation, the use of sequence-unspecific DNA labeling dyes (SYBR green), labeled primer-based technologies (e.g., AmpiFlour, Plexor, Lux), and techniques which involve double-labeled hybridization probes (e.g., Molecular Beacons) or hydrolysis probes (e.g., TaqMan, MGB, and LNA). Using any of these or other known chemistries, biomarker amplicon product generation is detected and measured by a suitable reading device. The reading device is typically a thermocyling device equipped with an optical sensing system that is capable of detecting labeled amplicon products when produced. The reader can be any of a variety of known devices, (e.g., Roche® Lightcycler® 480, Illumina® Eco RT-PCR Instrument, and Life Technologies® 7500 Fast Dx RT-PCR Instrument) that export the measured amplicon data to the statistical computing device 10 via communication network 22 or other suitable input.

Another approach for quantifying expression levels involves sequencing of cDNA fragments of interest and counting the number of times a particular fragment has been observed, e.g., the serial analysis of gene expression (SAGE) method and massively parallel signature sequencing (MPSS) method. Both methods involve the use of restriction enzymes to obtain short sequence fragments (tags), usually from the 3′ end of an mRNA, which are subsequently digitally counted. These methods have been adapted to next-generation sequencing platforms to provide a high-throughput, cost effective, and more reliable method of expression analysis. Next-generation variations of these methods that are suitable for use in the present invention include, DeepSAGE (Nielsen et al., ‘DeepSAGE—Digital Transcriptomic with High Sensitivity, Simple Experimental Protocol and Multiplexing of Samples,” Nucleic Acids Res. 34:e 133 (2006), which is hereby incorporated by reference in its entirety), rapid analysis of 5′ transcript ends (5′-RATE) (Gowda et al., “Robust Analysis of 5′-Transcript Ends (5′-RATE): A Novel Technique for Transcriptome Analysis and Genome Annotation,” Nucleic Acids Res. 34:E126 (2006), which is hereby incorporated by reference in its entirety), and Tag-Seq (Morrissy et al., “Next-Generation Tag Sequencing for Cancer Gene Expression Profiling,” Genome Res. 19:1825-35 (2009), which is hereby incorporated by reference in its entirety), among others. Next-generation sequencing platforms suitable for mRNA and microRNA expression analysis that can be utilized in carrying out the methods of the present invention include, without limitation, Applied Biosystem® SOLiD™ SAGE™ System, Roche® 454 GS-FLX technology, and Illumina® Genome Analyzer.

When the molecular biomarkers to be measured are protein expression levels or metabolite concentrations, a quantitative immuno-based or mass-spectrometry based assay can be employed to measure protein expression or metabolite concentration. By, way of example only, a suitable high-throughput immunoassay for proteomic analysis involves the use of an antibody microarray as described by Wingren et al., “High-throughput Proteomics Using Antibody Microarrays,” Expert Rev. Proteomics, 1(3): 355-64 (2004) and Uhlen et al., “Antibody-Based Proteomics for Human Tissue Profiling,” Mol. Cell. Proteomics 4:384-393 (2005), which are hereby incorporated by reference in their entirety). Suitable mass-spectrometry platform assays for proteomic analysis involve the use of MALDI-TOF mass spectroscopy (Pan et al., “High Throughput Proteome Screening for Biomarker Detection,” Mol. Cell. Proteomics 4: 182-190 (2005), which is hereby incorporated by reference in its entirety), Fourier transform-ion cyclotron resonance (FT-ICR) mass spectrometry (Smith et al., “An Accurate Mass Tag Strategy for Quantitative and High-Throughput Proteome Measurements,” Proteomic 2(5):513-523 (2002), which is hereby incorporated by reference in its entirety), and a high-efficiency multiple-capillary liquid chromatography system coupled to a FT-ICR mass spectrometer (Shen et al., “High-Throughput Proteomics Using High-Efficiency Multiple-Capillary Liquid Chromatography with On-line High-Performance ESI FTICR Mass Spectrometry,” Anal. Chem. 73(13): 3011-3021(2001), which is hereby incorporated by reference in its entirety). By way of example, suitable mass-spectrometry platform assays for metabolite analysis involve ultrahigh-field FTICR mass spectrometry (Han et al., “Towards High-Throughput Metabolomics using Ultrahigh-Field Fourier Transform Ion Cyclotron Resonance Mass Spectrometry,” Metabolomics 4(2):128-140 (2008), which is hereby incorporated by reference in its entirety), laser desorption ionization mass spectrometry (Vaidyanathan et al., “A Laser Desorption Ionisation Mass Spectrometry Approach for High Throughput Metabolomics,” Metabolomics 1(3):243-250 (2005), which is hereby incorporated by reference in its entirety), gas chromatography-mass spectrometry (GC/MS) (Jonsson et al., “High-Throughput Data Analysis for Detecting and Identifying Differences between Samples in GC/MS-based Metabolomic Analyses,” Anal. Chem. 77(17):5635-5642 (2005), which is hereby incorporated by reference in its entirety), and linear ion trap mass spectrometry (Koulman et al., “High-Throughput Direct-Infusion Ion Trap Mass Spectrometry: A New Method for Metabolomics,” Rapid Comm. Mass Spec. 21(3): 421-428 (2007), which is hereby incorporated by reference in its entirety).

In the methods of the invention, it is contemplated that multiple samples are obtained from each individual within a cohort of patients. As such, for each such patient two or more expression levels can be obtained. When there are multiple, differing expression levels of candidate molecular biomarkers associated with a single patient (and that patient's phenotype), it is only the minimum and maximum expression levels that will be obtained and analyzed in accordance with the present invention. When there are multiple, identical expression levels of the candidate molecular biomarker, then the minimum and maximum expression levels are the same for the patient. When there is only one sample obtained from a patient (i.e., there is only one tumor or disease lesion), then the minimum and maximum expression levels for a particular candidate molecular biomarker are the same for the patient.

In step 120, the statistical computing device 10 assesses statistically the association of the minimum and maximum expression levels of each candidate molecular biomarker obtained in step 110 with a disease phenotype. By way of example, the statistical computing device 10 performs a univariate analysis, such as a Cox proportional hazards analysis or any formal statistical hypothesis test suitable for inferring an association between expression levels and phenotype, to assess the statistical association of the minimum and/or maximum expression levels of a particular candidate molecular biomarker that correlate with disease phenotype.

In step 130, the statistical computing device 10 assigns p-values to the minimum and maximum expression levels that were determined to correlate with disease phenotype by the statistical assessment of step 120. The p-value is a measure of the correlation of a particular expression level for a biomarker to a disease phenotype.

In step 140, the statistical computing device 10 ranks the p-values assigned to each expression level from step 130 in order of most to least significant.

In step 150, the statistical computing device 10 selects the minimum and/or maximum expression levels of the candidate molecular biomarker that significantly correlate with disease phenotype among the patient cohort. This selection is based on the results of the assessment of statistical association of step 120, and subsequent assignment 130 and ranking of p-values 140 which indicate the significance of association between the measured expression level and the disease phenotype. As noted above, when analyzing a disease involving multiple disease samples which may be heterogenic in biomarker expression, it is preferable to measure the minimum and maximum expression levels of the candidate biomarkers across all samples obtained from the patient. This anticipates the possibility that a biomarker expression level which is predictive of a disease phenotype may be differentially expressed among the different tissue samples. For example, as described herein, hepatocellular carcinoma is frequently associated with multifocal intrahepatic disease, and examination of biomarker expression in each disease lesion is warranted for an accurate determination of disease phenotype. When the maximum and minimum expression values from more than one tissue sample from a subject are obtained from the patient database server 24 or measured by the statistical computing device 10, only the lowest minimum and highest maximum expression levels are selected by the statistical computing device 10 for correlation to disease phenotype.

In step 160, the statistical computing device 10 generates a standardized expression value (SEV) for each of the selected minimum and maximum expression levels. By way of example, generating a standardized expression value is carried out by the statistical computing device 10 according to the equation of formula (I):

Zi=(i−i_{median})/i_{IQR} (I)

where

Zi is the standardized expression value;

i is the minimum or maximum measured expression level of the molecular biomarker in an individual in the cohort of patients;

i_{median} is the median expression level of the molecular biomarker calculated across the cohort of patients;

i_{IQR} is the interquartile range (difference between the 75^{th} and 25^{th} percentiles) of expression level of the molecular biomarker calculated across all the cohort of patients; and

wherein

when Zi is <0, Zi is multiplied by −1.

Thus, the SEV for each minimum or maximum expression level is a positive number that is associated with a particular phenotype.

In step 170, the statistical computing device 10 constructs a biomarker reference score (“B_{RS}”) for each individual by summing the SEVs of the candidate molecular biomarkers whose inclusion in the sum maximizes the correlation between the biomarker reference score and the known disease phenotype of the individual. By way of example, step 170 is carried out using the statistical computing device 10 by initially setting B_{RS}=Z₁, where Z₁ is the SEV of the most significantly correlated minimum or maximum expression level as determined in step 140, i.e., the SEV with the most significant p-value. Then the following algorithm is employed:

For i=Z_{M} where M is the maximum biomarker index, do

Set B_{RS}*=B_{RS}+Zi

Calculate R² for both B_{RS}* and B_{RS}

If R² for B_{RS}* is higher than R² for B_{RS}, then set B_{RS}=B_{RS}* and repeat for Z₍ᵢ₊₁₎

If R² for B_{RS} is higher than R² for B_{RS}*, then do not change B_{RS}

Using this algorithm, the final B_{RS} is the sum of Zi whose inclusion in the sum maximizes the correlation between the biomarker reference score and the known disease phenotype.

In step 180, the statistical computing device 10 summarizes the reference score distributions across the cohort for each disease phenotype and estimates a biomarker reference threshold score (“B_{TS}”). Using the respective biomarker reference score distributions to determine the biomarker reference threshold score yields a satisfactory balance of specificity and sensitivity. In practice, selection of the reference threshold score may further involve consideration of various other criteria, and may incorporate information such as donor availability or auxiliary subject risk factors.

Following initial development and definition of a biomarker reference threshold score based on a particular cohort of patients, the biomarker reference threshold score can be validated using an independent dataset. As described herein in the Examples, across validation study incorporates all of the same steps used to identify and define the biomarker reference threshold score, the only difference is that a new cohort of patients is employed.

The present invention also relates to the clinical application of the defined biomarker, i.e., the defined biomarker reference threshold score that is maximally correlated to a specific disease phenotype. Clinical application of a defined biomarker involves determining a “risk score” for a patient whose disease phenotype is unknown. The risk score is calculated based on the sum of standardized expression levels of one or more molecular biomarkers in disease tissue from the patient, where the one or more molecular biomarkers were selected during biomarker identification as having expression levels that maximally correlate to a particular disease phenotype. The risk score for a subject is compared to the biomarker reference threshold score that was defined during biomarker identification as the threshold value distinguishing particular disease phenotypes to determine the risk or likelihood that the subject will develop a particular disease phenotype.

As described herein, applicants developed and carried out the above described methodology for identifying a biomarker of hepatocellular disease recurrence. The details of biomarker generation are described herein in the Examples. The generated biomarker has clinical utility for patients having hepatocellular carcinoma and their doctors who are trying to determine the best course of therapeutic intervention. Curative treatment is limited to surgical resection and liver transplantation. The prior art standard for determining, patient eligibility for transplantation is the Milan criteria (1 tumor≦5 cm, 2-3 tumors≦3 cm, and no evidence of intrahepatic vascular invasion or extrahepatic spread). However, these criteria are based primarily on radiographic characteristics and do not assess individual tumor biology and risk of recurrence or overall survival. As a result, there are some patients that do not meet the Milan criteria who have a low incidence of recurrence and, likewise, there are patients who meet the Milan criteria who do suffer from recurrent disease after transplant. The biomarker described herein is indicative of an individual tumor's propensity for recurrence and, therefore, is of great value for optimizing overall patient outcomes, especially when used in conjunction with the Milan criteria.

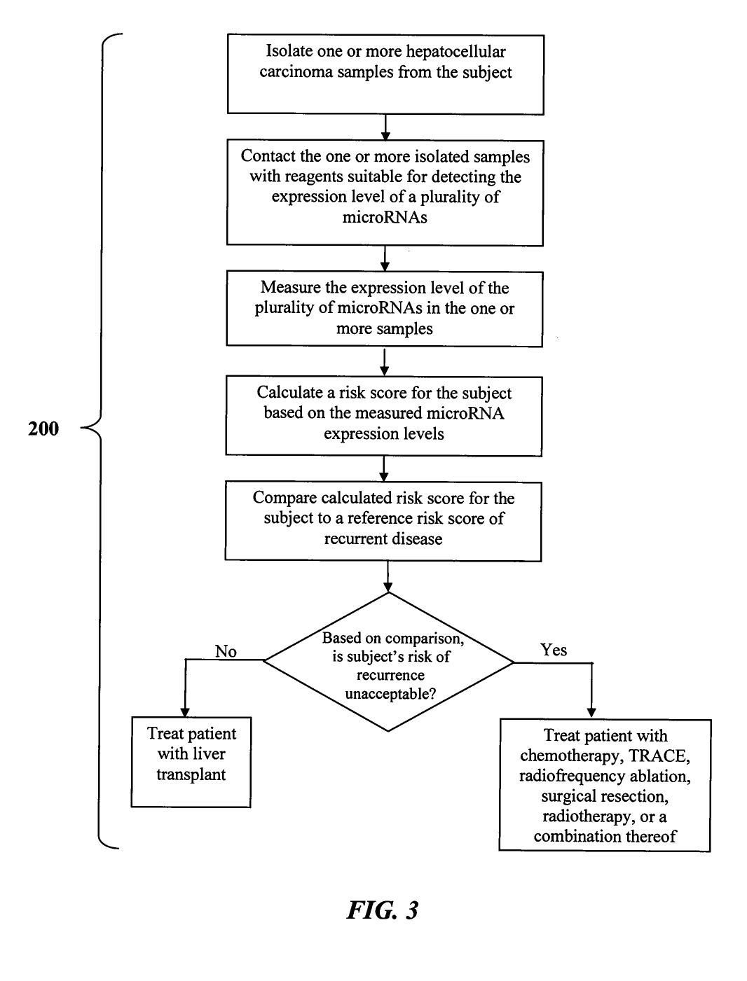

Accordingly, another aspect of the present invention is directed to a method of determining a subject's risk of developing recurrent hepatocellular carcinoma. This method involves contacting an isolated hepatocellular carcinoma sample from the subject with reagents suitable for detecting expression levels of two or more microRNAs in the sample and measuring the expression levels of the two or more microRNAs in the sample based on said contacting. The method further involves calculating a risk score of hepatocellular carcinoma disease recurrence for the subject based on the measured microRNA expression levels in the isolated sample and comparing the calculated risk score for the subject to a reference threshold score of hepatocellular disease recurrence to determine the subject's risk of developing recurrent hepatocellular carcinoma (HCC). A flow chart 200 depicting this method is shown in FIG. 3.

MicroRNAs (miRNAs) are a class of non-coding RNA molecules of about 19-25 nucleotides derived from endogenous genes which act as post-transcriptional regulators of gene expression. They are processed from longer (ca 70-80 nt) hairpin-like precursors termed pre-miRNAs by the RNAse III enzyme Dicer. miRNAs assemble in ribonucleoprotein complexes termed “miRNPs” and recognize their target sites by antisense complementarity, thereby mediating down-regulation of their target genes. Near-perfect or perfect complementarity between the miRNA and its target site results in target mRNA cleavage, whereas limited complementarity between the miRNA and the target site results in translational inhibition of the target gene. As used herein, microRNA or miRNA refers to the unprocessed or processed RNA transcript.

In accordance with this aspect of the invention, the two or more microRNAs are selected from the group of microRNAs consisting of hsa-miR-501, hsa-miR-1180, hsa-miR-365, hsa-miR-1273, hsa-miR-377, hsa-let-7d, hsa-miR-576, hsa-miR-454, hsa-miR-18a, hsa-miR-15a, hsa-miR-548c, hsa-miR-20a, hsa-miR-610, miR-146b, hsa-miR-137, hsa-miR-1293, hsa-miR-139, hsa-miR26a, hsa-miR-122, hsa-miR-192, hsa-miR-888, hsa-miR-497, hsa-miR-592, hsa-miR-545, hsa-miR-513a, hsa-miR-136, hsa-miR-1226, hsa-miR-651, hsa-miR-542, hsa-miR-491, hsa-miR-937, hsa-miR-424, hsa-miR-630, hsa-miR-33b, hsa-miR-615, hsa-mir-152, hsa-miR-455, hsa-miR-23b, hsa-miR-671, hsa-miR-30c-2, hsa-miR-193b, hsa-miR-1260, hsa-miR-505, hsa-miR-181c, hsa-miR-99a, hsa-miR-885, hsa-miR-145, hsa-miR-194, hsa-miR-125b-2, hsa-miR-182.

For purposes of the present invention, all forms of the aforementioned microRNAs, i.e., −5p, −3p, star, a, b, c, etc., are intended to be encompassed by the generic microRNA recited. The nucleotide sequences of the microRNA precursor molecules, i.e.

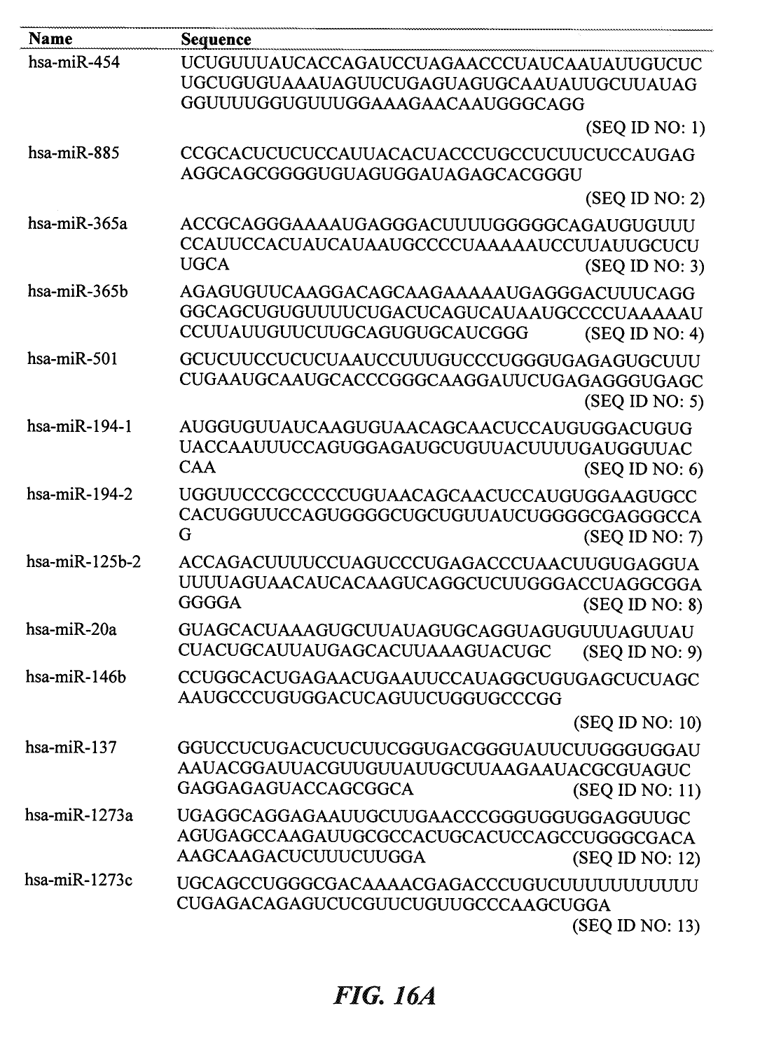

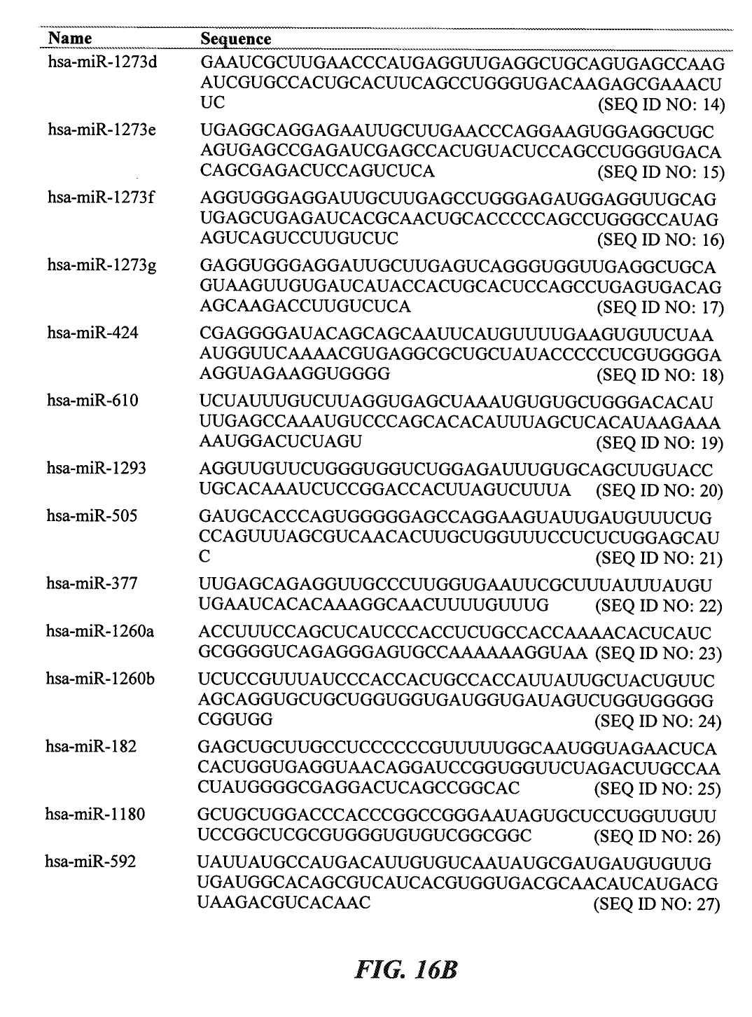

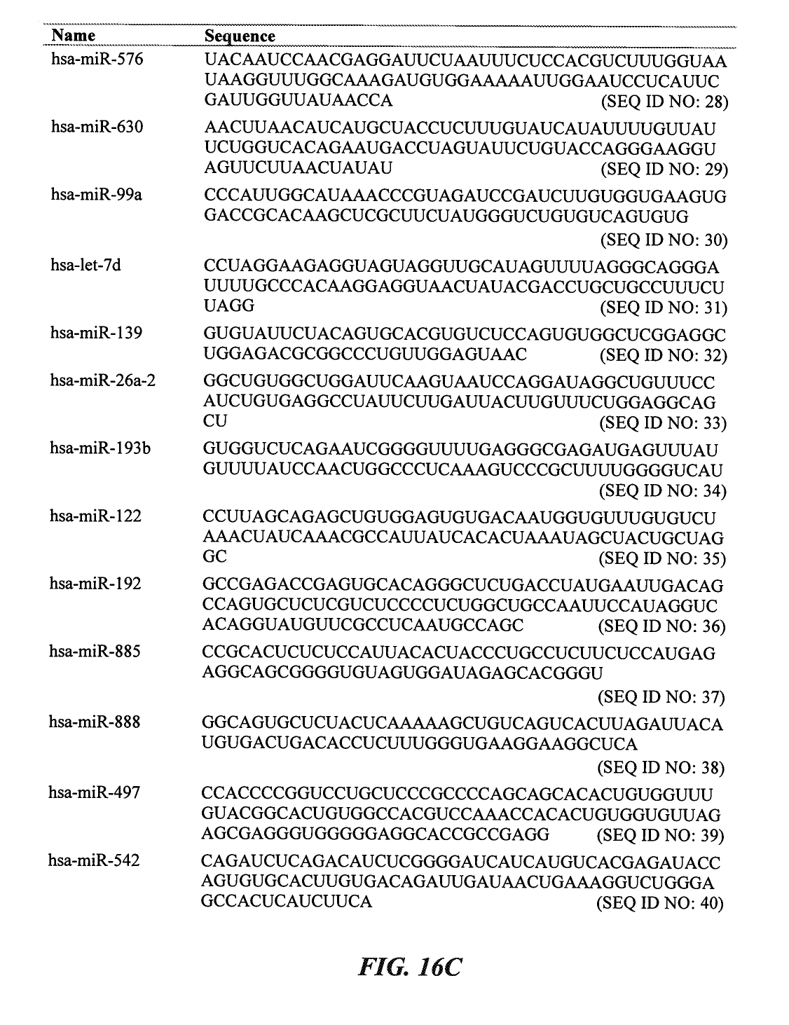

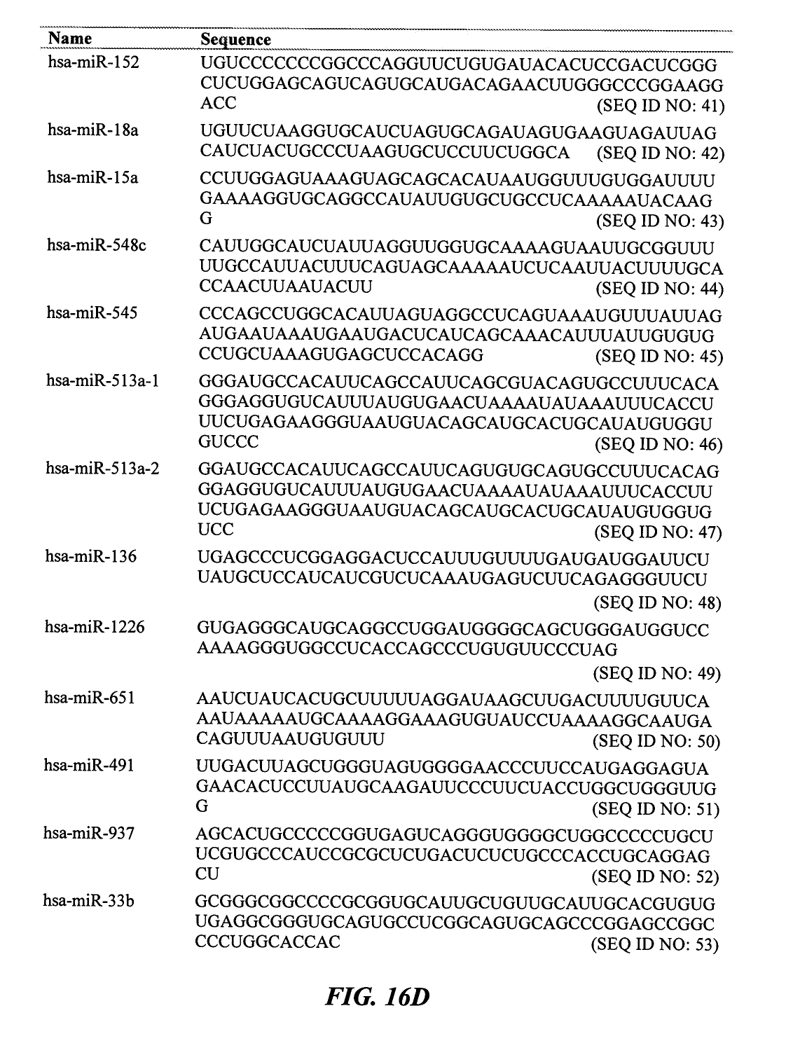

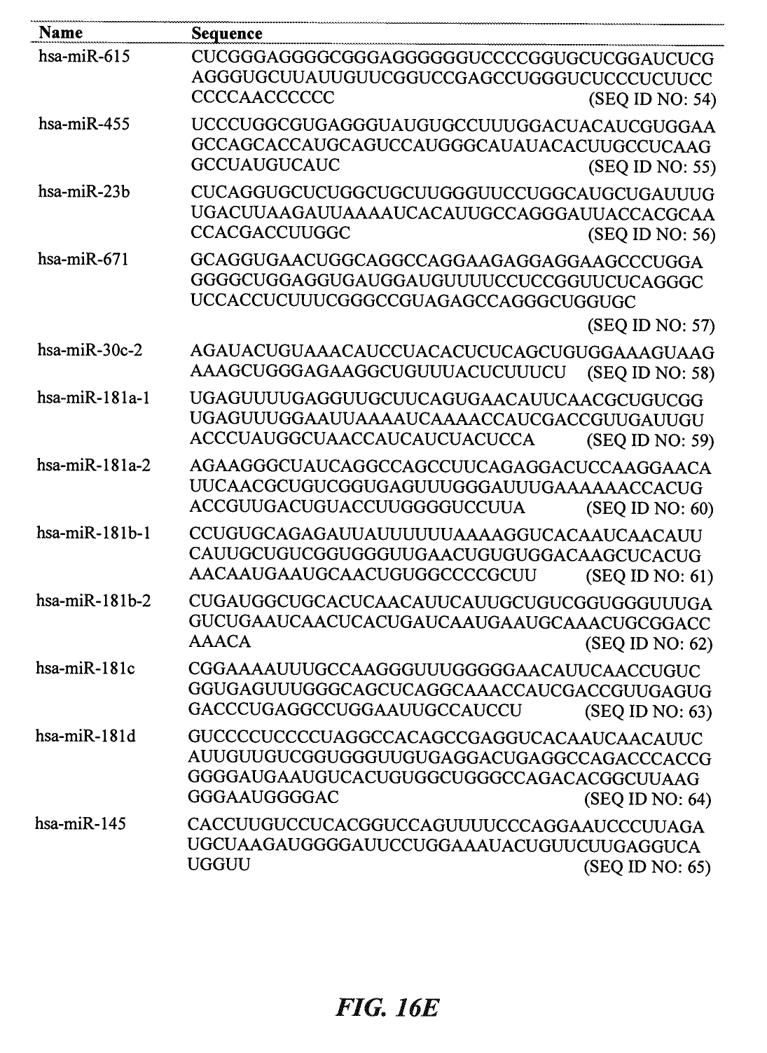

SEQ ID NOs: 1-65, are depicted in FIG. 16.

In one embodiment of the present invention, the maximum and minimum expression levels of all 50 of the microRNAs listed above are measured and used to calculate a patient's risk score of hepatocellular carcinoma disease recurrence. Alternatively, the expression levels of at least 45, at least 40, or at least 35 of the above listed microRNAs are measured and used to calculate a patient's risk score.

In one embodiment of this aspect of the present invention, the subject has one hepatocellular tumor. In this case, the maximum and minimum expression levels of the above noted microRNAs will be the same value. However, in another embodiment of this aspect of the present invention, the subject has more than one hepatocellular tumor. Samples from each tumor are obtained and the maximum and minimum expression levels for each of the above noted microRNAs are measured. The risk score of the latter individual is calculated based on the maximum and minimum expression levels, if different, that are measured.

In another embodiment of the present invention, the two or more microRNAs are selected form the group of microRNAs consisting of hsa-miR-454, hsa-miR-885, hsa-miR-365, hsa-miR-501, hsa-miR-194, hsa-miR-125b-2, hsa-miR-20a, hsa-miR-146b, hsa-miR-137, hsa-miR-1273, hsa-miR-424, hsa-miR-610, hsa-miR-1293, hsa-miR-505, hsa-miR-377, hsa-miR-1260, hsa-miR-182, hsa-miR-1180, hsa-miR-592, hsa-miR-576, hsa-miR-630, hsa-miR-99a, hsa-let-7d, hsa-miR-139, hsa-miR-26a-2, hsa-miR-193b, hsa-miR-122, hsa-miR-192, hsa-miR-885, hsa-miR-888, hsa-miR-497, hsa-miR-542, and hsa-miR-152.

In a preferred embodiment of the present invention, the maximum and minimum expression levels of all 33 of the microRNAs listed above are measured and used to calculate a patient's risk score of hepatocellular carcinoma disease recurrence. Alternatively, the expression levels of at least 30, at least 25, at least 20, at least 15, at least 10, or at least 5 of the most relevant microRNA expression levels of the above-listed 33 microRNAs are measured and used to calculate a patient's risk score.

The expression level of a particular microRNA product in a sample can be measured using any technique that is suitable for detecting RNA expression levels in a biological sample. Suitable techniques include hybridization, amplification, and next-generation sequencing based assays as described above.

Array-based methods for microRNA detection and quantitation are known in the art, see e.g., U.S. Pat. No. 7,635,563 to Horvitz et al., and Sioud et al., “Profiling microRNA Expression Using Sensitive cDNA Probes and Filter Arrays,” Biotechniques 37(4): 574-580 (2004) which are hereby incorporated by reference in their entirety. Human microRNA arrays for expression profiling are also commercially available (e.g., Exiqon, Vedbaek, Denmark, Affymetrix, Santa Clara, Calif., and Life Technologies,

Carlsbad Calif.). Using an array-based hybridization method involves isolating and, optionally, enriching for the RNA fraction of a heptocellular carcinoma sample taken from the patient. microRNA labeling can be carried out following the method described in U.S. Pat. No. 7,635,563 to Horvitz, which is hereby incorporated by reference in its entirety. Briefly, oligonucleotide linkers are attached to the 5′ and 3′ ends of the microRNAs using a ligation reaction and the resulting ligation products are used as templates for an RT-PCR reaction using fluorescently labeled PCR primers. Several other methods for labeling mRNA are known in the art and can be adapted for microRNA labeling for purposes of the present invention (see e.g., Duggan et al., “Expression Profiling using cDNA Microarrays,” Nat. Genet. 21:10-14 (1999); Schena et al., “Quantitative Monitoring of Gene Expression Patterns with a Complementary DNA Microarray,” Science 270:467-470 (1995); Nimmakayalu et al., “Simple Method for Preparation of Fluor/Hapten-Labeled dUTP,” Biotechniques 28:518-522 (2000); Gupta et al., “Directly Labeled mRNA Produces Highly Precise and Unbiased Differential Gene Expression Data,” Nucleic Acids Res 31:e13 (2003), which are hereby incorporated by reference in their entirety). The sample containing the labeled microRNA is subsequently contacted with a microarray of complementary oligonucleotide probes to facilitate detection. Conditions suitable for hybridization are optimized to limit or avoid non-specific binding between the probes and target microRNAs in the sample. Typically, the hybridization temperature is between 50-85° C.; however, optimization of hybridization conditions can be carried out as described in U.S. Pat. No. 7,635,563 to Horvitz, which is hereby incorporated by reference in its entirety.

In addition to array based hybridization assays described above, other suitable hybridization based assays include northern blot analysis, in-situ hybridization, solution hybridization, and RNAase protection assays, all of which are well known in the art. Any hybridization assay requires the use of one or more oligonucleotide probes that is complementary to at least a portion of the nucleotide sequence comprising the microRNA target sequence. Suitable probes for a hybridization based assay can be produced using the nucleotide sequences of SEQ ID NOs: 1-65 that are shown in FIGS. 16A-16E. Oligonucleotide probes suitable for detecting microRNA are typically between 19-25 nucleotides in length depending on the length of the target microRNA being detecting. Methods for preparing labeled oligonucleotide probes and the conditions for hybridization thereof to target microRNA nucleotide sequences are known in the art and described, for example, in MOLECULAR CLONING: A LABORATORY MANUAL (J Sambrook et al., eds., Cold Spring Harbor Laboratory Press, 1989), which is hereby incorporated by reference in its entirety.

As described above, a suitable amplification-based assay is a quantitative real-time PCR assay. Other suitable amplification-based assays include, but are not limited to, ligation chain reaction (LCR), transcription amplification, self-sustained sequence replication, dot PCR, and linker adapted PCR, all of which are well known in the art. Amplification-based assays require preparing an isolated or enriched sample of RNA or cDNA from the hepatocellular sample obtained from the subject. Methods of

RNA purification and, optionally, reverse transcription to prepare a sample of cDNA are well known in the art. The sample is subsequently contacted with the oligonucleotide primers and in some cases one or more oligonucleotide probes (e.g., TaqMan-based assays), along with a polymerase enzyme and suitable amplification reaction buffers. Suitable primers and probes for the amplification and measurement of microRNA expression levels can be designed using the nucleotide sequences of SEQ ID NOs: 1-65 that are shown in FIGS. 16A-16E. Methods for primer/probe design and real-time quantitation of microRNA are well known in the art, see e.g., Shi et al., “Facile Means for Quantifying microRNA Expression by Real-Time PCR,” BioTechniques 39:519-525 (2005), Chen et al., “Real-Time Quantification of microRNAs by Stem-Loop RT-PCR,” Nucleic Acid Res. 33(20):e179 (2005); Raymond et al., “Simple, Quantitative Primer-Extension PCR Assay for Direct Monitoring of microRNAs and Short-Interfering RNAs,” RNA 11(10:1737-44 (2005), which are hereby incorporated by reference in their entirety. In addition, various commercial companies offer primer/probe design and validation to accompany their amplification chemistries. e.g., TaqMan assays by Applied Biosystems employ predesigned and validated stemloop primers.

As described herein, establishing a reliable biomarker for diseases such as HCC that have multiple tumors is problematic using prior art approaches. Previous attempts to define a microRNA biomarker signature for HCC are inadequate for a number of reasons. Firstly, they compare microRNA expression levels in tumor tissue and non-tumor tissue. This type of analysis does not identify differential microRNA expression between tumors having the propensity to recur and those lacking this propensity. In addition, these previous studies either look at expression levels in only one tumor or average the expression levels across multiple tumors. This approach ignores tumor heterogeneity and, as a result, will fail to properly identify microRNA expression that distinguishes recurrent from nonrecurrent disease. In contrast, the method described herein accounts for tumor variability by measuring the minimum and maximum microRNA expression levels in two or more tumor samples from a patient having multiple disease lesions. The risk score for the subject is subsequently calculated based on both of the measured minimum and maximum microRNA expression levels, if different, in the isolated samples.

A “risk score of hepatocellular disease recurrence” represents the risk or likelihood that an individual patient having hepatocellular carcinoma will develop recurrent disease. This risk score is calculated for an individual patient (whose disease phenotype is unknown) based on the sum of standardized minimum and maximum expression levels of the aforementioned microRNA in one or more disease samples obtained from the subject. This score is subsequently compared to a “reference threshold score of recurrent hepatocellular carcinoma” that, as shown herein, is the threshold value that distinguishes recurrent and non-recurrent disease phenotypes in a large cohort of patients.

A “reference threshold score of recurrent hepatocellular carcinoma” is defined based on the expression levels of the same microRNAs measured in the sample(s) from the individual patient. The development of a suitable reference threshold score from a cohort of recurrent and non-recurrent HCC patients is described herein in the Examples. The biomarker development process yielded separate and observable sample distributions of reference scores of standardized microRNA expression levels for subjects with both the recurrent and non-recurrent phenotype. The reference threshold value was defined based on the sample distributions of these reference scores, and provides a threshold value from which a prediction of recurrence can be made for an individual based on their calculated risk score of hepatocellular carcinoma disease recurrence. This reference threshold score is selected to yield an optimal balance of false positives (non-recurrent subjects with risk scores above the threshold) and false negatives (recurrent subjects with risk scores below the threshold). Application of the biomarker development process to the cohort of patients as described herein yielded a reference threshold score of approximately 3.8. This score was selected using a 90% specificity criterion (i.e., false negative=10%). Selection of the threshold score using different specificity or sensitivity criterion will alter the exact value of the score. Additionally, cross validation studies and expansion of the cohort will also alter the exact value of the reference threshold score.

In clinical application, for an individual patient whose disease phenotypes is unknown, a risk score of hepatocellular carcinoma disease recurrence for that patient that is higher than the reference threshold score of hepatocellular carcinoma disease recurrence for the cohort indicates the subject is likely to develop recurrent disease. In contrast, a risk score of an individual patient that is lower than the reference threshold score of the cohort indicates the subject is not likely to develop recurrent disease.

As noted above, the first step in calculating an individual's risk score is to standardize the measured minimum and maximum microRNA expression levels for the subject. A preferred approach for standardizing the measured expression level is to utilize the equation of formula (I):

Zi=(i−i_{median})/i_{IQR} (I)

where

Zi is the standardized expression value;

i is the minimum or maximum measured expression level of the microRNA in an individual in the cohort of patients;

i_{median} is the median expression level of the microRNA calculated across the cohort of patients;

i_{IQR} is the interquartile range of expression level of the microRNA calculated across the cohort of patients; and

wherein

when Zi is <0, Zi is multiplied by −1.

Once the microRNA expression levels of the subject are standardized, they are summed to form a final risk score and compared to the reference threshold score as described above.

The subject's risk of developing recurrent HCC disease as determined using the methods of the present invention is useful for selecting and administering a proper course of treatment for an individual. However, it is also desirable to consider other prognostic criteria, such as the Milan criteria, when making treatment decisions. As noted above, the Milan criteria considers cancer lesion size, number, and the presence of extrahepatic manifestations and vascular invasion. In accordance with this embodiment of the present invention, the subject's risk score of hepatocellular carcinoma disease recurrence is determined based on the combination of the summed standardized microRNA expression levels and additional prognostic criteria. In some cases, a patient may not meet the Milan criteria for receiving a liver transplant. However, if the subject has a low risk of developing recurrent disease based on microRNA expression levels, then the patient may still be a good candidate for liver transplant. In cases where the patient meets the Milan criteria but has a high risk of developing recurrent disease, then the medical professionals will need to make an assessment of the likelihood of recurrent disease (i.e., based on how close to the threshold score the patient is) and the Milan criteria to decide how best to proceed. In cases where the patient does meet the Milan criteria for receiving a liver transplant, and has a high risk of developing recurrent disease based on microRNA expression levels, alternative therapeutic strategies, such as transcatheter arterial chemoembolization, radiofrequency ablation, surgical resection, radiotherapy, chemotherapy, or some combination thereof may be administered in lieu of performing a liver transplant.

Another aspect of the present invention is directed to a method of treating a subject having hepatocellular carcinoma. This method involves calculating the subject's risk score for developing recurrent hepatocellular carcinoma based on measured expression levels of two or more microRNAs in one or more isolated hepatocellular carcinoma samples from the subject and comparing the calculated risk score for the subject to a reference threshold score of disease recurrence. A suitable therapy for the subject is administered based on the calculated risk score of hepatocellular carcinoma disease recurrence. This approach is illustrated in FIG. 3.

The method of determining a subject's risk score for developing recurrent disease and suitable therapies to administer based on this risk score are described supra.

Another aspect of the present invention is directed to a kit comprising a collection of oligonucleotides, said collection consisting essentially of two or more oligonucleotides that hybridize under stringent conditions to two or more microRNAs, respectively, wherein the two more microRNAs are selected from the group of hsa-miR-501, hsa-miR-1180, hsa-miR-365, hsa-miR-1273, hsa-miR-377, hsa-let-7d, hsa-miR-576, hsa-miR-454, hsa-miR-18a, hsa-miR-15a, hsa-miR-548c, hsa-miR-20a, hsa-miR-610, miR-146b, hsa-miR-137, hsa-miR-1293, hsa-miR-139, hsa-miR26a, hsa-miR-122, hsa-miR-192, hsa-miR-888, hsa-miR-497, hsa-miR-592, hsa-miR-545, hsa-miR-513a, hsa-miR-136, hsa-miR-1226, hsa-miR-651, hsa-miR-542, hsa-miR-491, hsa-miR-937, hsa-miR-424, hsa-miR-630, hsa-miR-33b, hsa-miR-615, hsa-mir-152, hsa-miR-455, hsa-miR-23b, hsa-miR-671, hsa-miR-30c-2, boo-miR-193b, hsa-miR-1260, hsa-miR-505, hsa-miR-181c, hsa-miR-99a, hsa-miR-885, hsa-miR-145, hsa-miR-194, hsa-miR-125b-2, hsa-miR-182.

In one embodiment of this aspect of the present invention, the kit is suitable for evaluating the expression of the above noted microRNAs using an array. In accordance with this embodiment of the present invention, the kit may include reagents for isolating microRNA, labeling microRNA, and/or evaluating the miRNA population using an array. The kit may further include reagents for creating or synthesizing microRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the microRNA probes, and components for isolating miRNA. Other kits may include components for making a nucleic acid array comprising oligonucleotides complementary to miRNAs, and thus, may include, for example, a solid support. Also, in certain embodiments, control RNA or DNA can be included in the kit. The control RNA can be miRNA that can be used as a positive control for labeling and/or array analysis.

In another embodiment of this aspect of the present invention, the kit is suitable for evaluating the expression of the above noted microRNAs using an amplification assay. In accordance with this embodiment of the present invention, the kit may include reagents for isolating microRNA, primers and/or probes suitable for amplifying the microRNAs of interest and optionally labeled, a reverse transcriptase, a polymerase, dNTPs, and one or more amplification reaction buffers.

For any kit embodiment of the present invention, a collection of oligonucleotide that hybridize under stringent conditions to two or more of the microRNAs listed above. These oligonucleotides contain a sequence that is identical to or complementary to all or part of any of the microRNA sequences of SEQ ID NOs: 1-65 that are shown in FIG. 16. The collection of oligonucleotides may comprise at least 10, 15, 20, 25, 30, 35

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components) may be separately placed. However, various combinations of components may be comprised in a vial. The kits of the present invention also will typically include a means for containing the nucleic acids, and any other reagent containers in close confinement for commercial sale.

When the components of the kit are provided in one and/or more liquid solutions, the liquid solution is an aqueous solution, with a sterile aqueous solution being one preferred solution. Other solutions that may be included in a kit are those solutions involved in isolating and/or enriching miRNA from a mixed sample.

The kit can also include instructions for employing the kit components as well the use of any other reagent not included in the kit. Preferably, the kit includes instructions for using the collection of oligonucleotides, i.e., primers and/or probes to measure microRNA expression levels and a computer readable medium having stored thereon, instructions for defining a risk score of recurrent hepatocellular carcinoma based on the measured microRNA expression levels. Instructions may include variations that can be implemented.

### EXAMPLES

The following examples are provided to illustrate embodiments of the present invention but are by no means intended to limit its scope.

### Materials and Methods for Examples 1-6

Patient Cohort Description:

This study was performed with approval of the University of Rochester Research Subjects Review Board (RSRB00029467). A total of 95 tumor nodules were studied from 69 patients who underwent liver transplantation for HCC at the University of Rochester Medical Center between 1996 and 2008. Forty patients had recurrent HCC within 3 years of transplant and 29 had no recurrent disease within 3 years. Patients were well-matched with regards to etiology, gender, race, age, and HCV antibody status (Table 1). Patients in the nonrecurrent group tended to have lower grade tumors, earlier tumor stage and less vascular invasion compared to the recurrent group, but there was significant overlap in these characteristics between groups (Table 1). In the nonrecurrent group, 17 of 29 patients were within Milan criteria and in the recurrent group, 11 of 40 patients were within Milan. Milan criteria for this cohort were determined by pathologic evaluation of explanted livers at the time of transplant. In the recurrent group, patients were more likely to have multifocal disease (42.5% vs. 17.2%), to be outside Milan criteria (72.5% vs. 41.4%), to have more advanced HCC stage and less differentiated tumor grade, to have vascular invasion (70% vs. 24.1%) and larger mean tumor size.

TABLE 1
Cohort demographics for patients that received
liver transplants for HCC (n = 69).

| | | HCC Recurrence s/p OLT | |
|---|---|---|---|
| | | No Recurrence | Recurrence |
| | | (n = 29) | (n = 40) |
| Etiology | HCV | 13 | 21 |
| | HBV | 3 | 3 |
| | Laennec's | 3 | 5 |
| | NASH | 3 | 5 |
| | Other* | 7 | 6 |
| Age at Time of Transplant (Mean) | | 58.8 | 57.5 |
| Sex (Recipient) | F | 7 | 5 |
| | M | 22 | 35 |
| Recipient Race | African American | 1 | 2 |
| | Caucasian | 25 | 37 |
| | Hispanic | 0 | 1 |
| | Native American | 2 | 0 |
| HCV (Ab) | Neg | 13 (44.8%) | 19 (47.5%) |
| | Pos | 16 (44.8%) | 21 (52.5%) |
| Number of Tumors | n = 1 | 12 (41.4%) | 12 (30%) |
| | n = 2 | 6 (20.7%) | 3 (7.5%) |
| | n = 3 | 6 (20.7%) | 8 (20.0%) |
| | N > 3 | 5 (17.2%) | 17 (42.5%) |
| Milan Criteria | Within | 17 (58.6%) | 11 (27.5%) |
| | outside | 12 (41.4%) | 29 (72.5%) |
| Explant cancer stage | I | 9 | 6 |
| AJCC/UICC | II | 15 | 11 |
| 2002 6th Edition | IIIA | 4 | 22 |
| (Native LiverBx) | IIIB | 1 | 1 |
| Tumor Grade | Well Differentiated | 17 | 8 |
| | Well-Moderately | 4 | 2 |
| | Differentiated | | |
| | Moderately | 6 | 20 |
| | Differentiated | | |
| | Moderately-Poorly | 0 | 4 |
| | Differentiated | | |
| | Poorly | 1 | 5 |
| | Differentiated | | |

| | | |
|---|---|---|
| Vascular Invasion | 7 (24.1%) | 28 (70%) |
| Largest (Mean) Tumor Size (cm) | 3.6 | 5.3 |

*Hemachromatosis, Autoimmune Hepatitis, Biliary Atresia, Cryptogenic Cirrhosis

Histological Confirmation of Tumor Specimens:

Representative H&E sections of the formalin-fixed paraffin embedded (FFPE) blocks from the explanted tumors were reviewed by a pathologist to ensure presence of >70% viable tumor. Tissue cores (7 mm diameter) were then obtained from the corresponding blocks and re-embedded for further processing and miRNA isolation.

miRNA Purification and Array Hybridization:

miRNA was isolated from FFPE liver tumor tissues using the Roche High Pure miRNA isolation kit (Roche Diagnostics, Mannheim, Germany). MiRNA extraction was performed from individual tissue blocks using seven sections of 10 microns each. One to three extractions were performed for each tumor to generate sufficient miRNA for microarray analysis. All samples were assessed for presence of enriched miRNA using an Experion Bioanalyzer (Bio-Rad, Hercules, Calif., USA). MiRNAs were labeled using the FlashTag Biotin RNA labeling kit (Genisphere, Hatfield, Pa., USA) according to the provided protocol and then hybridized to Affymetrix GeneChipmiRNA 1.0 microarrays (Affymetrix, Santa Clara, Calif., USA). These arrays are comprised of 46 228 probe sets representing over 6703 miRNA sequences (7) organisms) from the Sanger miRNA database (V.11) and an additional 922 sequences of human snoRNA and scaRNA from the Ensemble database and snoRNABase. Array hybridization, washing and staining was performed at the Upstate Medical University microarray core facility in Syracuse, N.Y., per the manufacturer's instructions and arrays were scanned with a GeneChip Scanner 7G Plus. Data files (.cel files) were generated using the miRNA-1_{—}0_{—}2X gain library file. Hybridization quality metrics were assessed using the AffyMir miRNA QCTool program (version 1.0.33.0, Affymetrix, Santa Clara, Calif., USA). Only human miRNAs were considered in this analysis. All arrays were preprocessed using Robust Multiarray Average (RMA; Hochreiter et al., “A New Summarization Method for Affymetrix Probe Level Data,” Bioinformatics 22:943-949 (2006), which is hereby incorporated by reference in its entirety). RMA was performed on all 46,228 probe sets after which nonhuman probe sets were removed leaving 847 human miRNA probe sets. All data have been deposited onto the Gene Expression Omnibus (GEO accession number: GSE30297).

Description of the Min-Max Procedure for Biomarker Construction with Multifocal Tissue Samples:

The preprocessed data took the form of miRNA expression estimates for each feature, miR-X, in each sample. In the case of multifocal tissue samples, two or more samples were obtained from a single patient. To create a biomarker for patient prognosis, the miRNA expression estimates from each collection of samples belonging to the same patient were combined. This was done by constructing two new probe features, miR-X_MIN and miR-X_MAX, defined as the minimum and maximum expression (of miR-X) for each patient. As it cannot be generally anticipated whether high or low expression is associated with recurrence, miR-X_MIN and miR-X_MAX were treated as separate features in biomarker selection. Clearly, the MIN and MAX features were identical for unifocal patients. Although both the MIN and MAX features for a given miRNA can be statistically significant in a univariate analysis, at most one from each pair in any final biomarker will be used.

Statistical Analysis:

Array quality was assessed using a suite of widely used quality measures (Kauffmann et al., “ArrayQualityMetrics—A Bioconductor Package for Quality Assessment of Microarray Data,” Bioinformatics 25:415-416 (2009), which is hereby incorporated by reference in its entirety). The miR-X_MIN and miR-X_MAX features were constructed as previously described. Hierarchical clustering (Euclidean distance with complete agglomeration: see Gordon A., Classification Methods for the Exploratory Analysis of Multivariate Data. London:Chapman and Hall (1981), which is hereby incorporated by reference in its entirety) was used to assess both similarity of expression within subjects and within recurrence status. The primary outcome was defined as recurrence free survival time. The observation time of recurrence free subjects was, therefore, considered a right-censored survival time. The ability of each feature to predict survival time was assessed using a univariate Cox proportional hazards model. The resulting p value was interpreted as a measure of the feature's association with recurrence. The false discovery rate (FDR) adjusted p values were estimated using the Benjamini-Hochberg procedure (Benjamini Y., “Controlling the False Discovery Rate: A Practical and Powerful Approach to Multiple Testing,” J. R. Statist. Soc. B 57:289-300 (1995), which is hereby incorporated by reference in its entirety).

Standardized Scale Model (SSM) for Biomarker Identification:

A problem inherent in the development of biomarkers is the tendency for multivariate models to overfit data. This results in biomarkers that are initially reported to perform extremely well but whose performance cannot be reproduced. This tendency can be controlled to some degree using cross validation (CV). However, the unpredictability of multivariate models remains a problem, particularly when individual features exhibit a high degree of correlation. To address this concern, the following Standardized Scale Model for biomarker generation was used:

(1) For each feature, fit a univariate Cox proportional hazards model and record the p value and direction of association. Positive association means that greater feature expression yields longer recurrence free survival and negative association, the opposite.

(2) Using the p values from step (1), rank the features from most to least significant. Retain only a fixed number of the most significant features. The optimal number of features to retain was investigated during CV.

(3) Create a survival score by robustly standardizing the expression estimate of each retained feature by subtracting the median and dividing by the interquartile range (IQR), where the median and IQR are calculated across patients for each feature. For features whose direction of association from step (1) was negative, reverse the sign of the survival score (i.e., multiply by −1) so that a higher score is always associated with greater survival.

(4) Define an initial biomarker as the survival score of the most significant feature from step (2). Then proceed down the list of features from step (2), moving from more to less significant. For each feature, define a new (potentially improved) biomarker as the current biomarker plus the survival score of that feature from step (3). Compare the performance of the new biomarker to the current biomarker (assessed by the coefficient of variation (R2) from a Cox regression) and keep the better one.

(5) The final biomarker in step (4) will be the sum of the survival scores of those features that improved performance. Additional predictor(s), such as the Milan criteria, can be easily incorporated into this procedure by adding the additional predictor(s) to the Cox regression models in steps (1) and (4).

Cross Validation: To assess the predicted performance of a biomarker in an independent data set, a CV procedure was performed that incorporated all steps used for 56 subjects, and testing was performed on the remaining 8 subjects (the total number of subjects available was 64). Within each CV sample, a new biomarker was created (following the steps described above) and biomarker scores were obtained for all subjects. The quantiles of the biomarker scores for the test subjects constituted the CV prediction. This procedure was repeated 500 times yielding 8×500=4000 total CV predictions. Four types of biomarkers were evaluated, defined by how the samples were combined for multifocal patients (MIN-MAX or mean) and whether the Milan criteria was included as an additional predictor. For each model, a receiver operator characteristics (ROC) curve was plotted. The area under curve (AUC) is a straightforward way to assess the performance of a given biomarker.

### Example 1

### Quality Assessment of miRNA Purified from FFPE Tissues

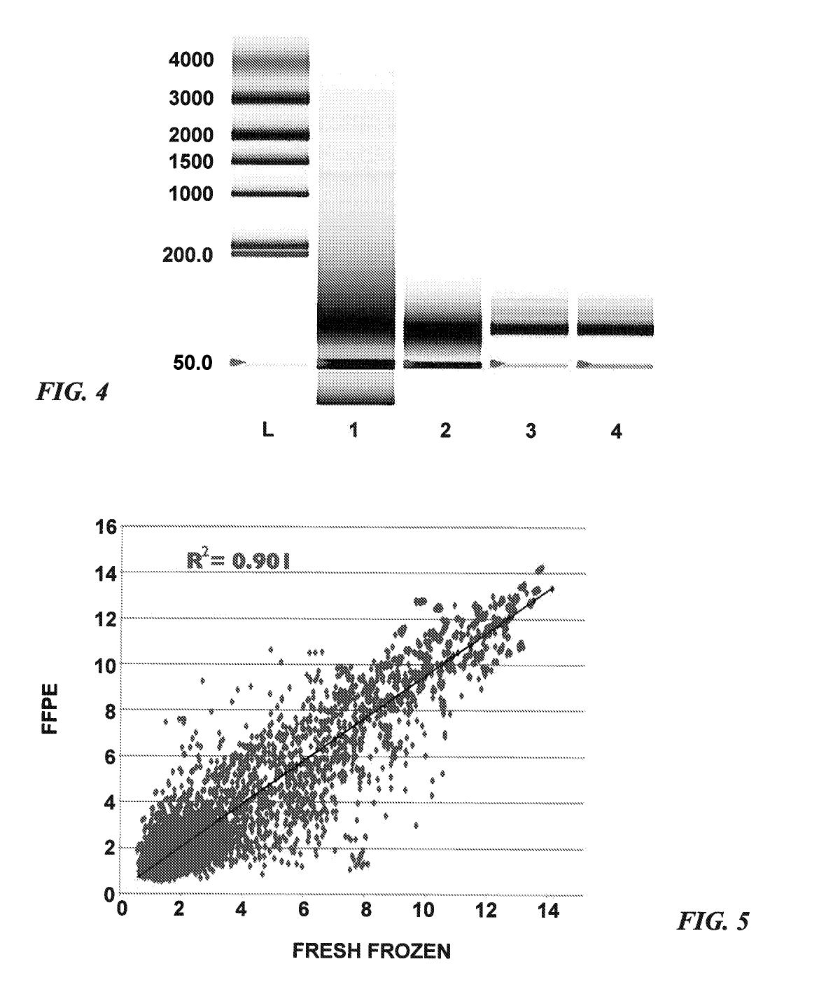

High yields of miRNA were consistently obtained from FFPE blocks based on electrophoresis and spectroscopy (FIG. 4). Furthermore, when miRNA obtained from freshly frozen cell lines was hybridized to the arrays and these results were compared to array hybridization of miRNA from the same cell lines that had first been FFPE, an excellent correlation was noted (R2=0.88-0.90, FIG. 5).

### Example 2

### Quality Metrics and Univariate Analysis

Seven arrays were removed due to poor quality as assessed by one of the six quality metrics considered. This resulted in 88 samples and 64 subjects for further analysis. The MIN-MAX procedure yielded 1694 features based on 847 probes. The univariate analysis yielded 60 significant features at 20% FDR (Table 2). A majority of the miRNAs distinguishing recurrence from nonrecurrence have been shown by others (Budhu et al., “Identification of Metastasis-Related MicroRNAs in Hepatocellular Carcinoma,” Hepatology 47:897-907 (2008); Sato et al., “MicroRNA Profile Predicts Recurrence After Resection in Patients With Hepatocellular Carcinoma Within the Milan Criteria,” PLoS One 6:e16435 (2011), which are hereby incorporated by reference in their entirety) to be relevant to hepatocellular carcinogenesis. One may expect both the MIN and MAX feature for some probes to be significant, particularly when there tends to be smaller variation within a multifocal sample, and the two features would then be approximately equal. This effect was relatively small in this cohort. The 60 significant features represented 50 distinct probe sets (of which 10 were represented by both MIN and MAX features). In Table 2, MIN and MAX represent the minimum and maximum expression probe features for a given miRNA. Highlighted probes are those selected between 70-97% in cross validation.

TABLE 2
Univariate analysis of miRNAs significant for
recurrent HCC within 3 years of transplant.

| Rank | Probe Features | unadj p-value | FDR |
|---|---|---|---|
| 1 | hsa-miR-194_st_MIN | 1.6E−06 | 0.003 |
| 2 | hsa-miR-454-star_st_MAX | 3.8E−05 | 0.024 |
| 3 | hsa-miR-125b-2-star_st_MIN | 4.9E−05 | 0.024 |
| 4 | hsa-miR-122_st_MIN | 5.7E−05 | 0.024 |
| 5 | hsa-miR-182_st_MIN | 1.8E−04 | 0.047 |
| 6 | hsa-miR-365_st_MAX | 2.0E−04 | 0.047 |
| 7 | hsa-miR-99a-star_st_MIN | 2.1E−04 | 0.047 |
| 8 | hsa-miR-192_st_MIN | 2.4E−04 | 0.047 |
| 9 | hsa-miR-885-5p_st_MIN | 2.5E−04 | 0.047 |
| 10 | hsa-miR-888_st_MAX | 2.8E−04 | 0.047 |
| 11 | hsa-miR-22_st_MIN | 5.4E−04 | 0.084 |
| 12 | hsa-miR-1274b_st_MIN | 7.5E−04 | 0.099 |
| 13 | hsa-miR-497_st_MIN | 8.1E−04 | 0.099 |
| 14 | hsa-miR-501-5p_st_MAX | 8.6E−04 | 0.099 |
| 15 | hsa-miR-542-5p_st_MIN | 8.9E−04 | 0.099 |
| 16 | hsa-miR-152_st_MIN | 9.4E−04 | 0.099 |
| 17 | hsa-miR-505_st_MIN | 0.0011 | 0.107 |
| 18 | hsa-miR-130a_st_MIN | 0.0012 | 0.114 |
| 19 | hsa-miR-885-5p_st_MAX | 0.0015 | 0.124 |
| 20 | hsa-miR-137_st_MAX | 0.0015 | 0.124 |
| 21 | hsa-miR-212_st_MIN | 0.0015 | 0.124 |
| 22 | hsa-miR-192-star_st_MIN | 0.0016 | 0.125 |
| 23 | hsa-miR-100_st_MIN | 0.0017 | 0.126 |
| 24 | hsa-miR-1273_st_MAX | 0.0020 | 0.138 |
| 25 | hsa-miR-122_st_MAX | 0.0020 | 0.138 |
| 26 | hsa-miR-571_st_MAX | 0.0021 | 0.138 |
| 27 | hsa-miR-935_st_MAX | 0.0026 | 0.164 |
| 28 | hsa-miR-139-5p_st_MIN | 0.0027 | 0.164 |
| 29 | hsa-miR-1260_st_MIN | 0.0030 | 0.168 |
| 30 | hsa-miR-377-star_st_MAX | 0.0031 | 0.168 |
| 31 | hsa-miR-99a_st_MIN | 0.0034 | 0.168 |
| 32 | hsa-miR-146b-3p_st_MIN | 0.0035 | 0.168 |
| 33 | hsa-miR-125b-2-star_st_MAX | 0.0035 | 0.168 |
| 34 | hsa-miR-372_st_MAX | 0.0035 | 0.168 |
| 35 | hsa-miR-224_st_MIN | 0.0036 | 0.168 |
| 36 | hsa-miR-186_st_MAX | 0.0036 | 0.168 |
| 37 | hsa-miR-148a_st_MIN | 0.0037 | 0.168 |
| 38 | hsa-miR-576-3p_st_MAX | 0.0041 | 0.183 |
| 39 | hsa-miR-1293_st_MAX | 0.0043 | 0.183 |
| 40 | hsa-miR-505_st_MAX | 0.0043 | 0.183 |
| 41 | hsa-miR-485-3p_st_MAX | 0.0048 | 0.197 |
| 42 | hsa-miR-610_st_MAX | 0.0049 | 0.197 |
| 43 | hsa-miR-671-3p_st_MIN | 0.0051 | 0.197 |
| 44 | hsa-miR-20a-star_st_MIN | 0.0053 | 0.197 |
| 45 | hsa-miR-132_st_MIN | 0.0053 | 0.197 |
| 46 | hsa-miR-422a_st_MIN | 0.0055 | 0.197 |
| 47 | hsa-miR-518d-3p_st_MAX | 0.0055 | 0.197 |
| 48 | hsa-miR-99a-star_st_MAX | 0.0058 | 0.197 |
| 49 | hsa-let-7d-star_st_MAX | 0.0059 | 0.197 |
| 50 | hsa-miR-373_st_MAX | 0.0061 | 0.197 |
| 51 | hsa-miR-224_st_MAX | 0.0063 | 0.197 |
| 52 | hsa-miR-147b_st_MAX | 0.0063 | 0.197 |
| 53 | hsa-miR-129-3p_st_MAX | 0.0064 | 0.197 |
| 54 | hsa-miR-505-star_st_MIN | 0.0065 | 0.197 |
| 55 | hsa-miR-501-5p_st_MIN | 0.0066 | 0.197 |
| 56 | hsa-miR-143-star_st_MIN | 0.0067 | 0.197 |
| 57 | hsa-miR-30a-star_st_MIN | 0.0068 | 0.197 |
| 58 | hsa-miR-1273_st_MIN | 0.0069 | 0.197 |
| 59 | hsa-miR-20a-star_st_MAX | 0.0069 | 0.197 |
| 60 | hsa-miR-365_st_MIN | 0.0070 | 0.197 |

### Example 3

### Unsupervised Hierarchical Clustering Results are Refined Using MIN-MAX

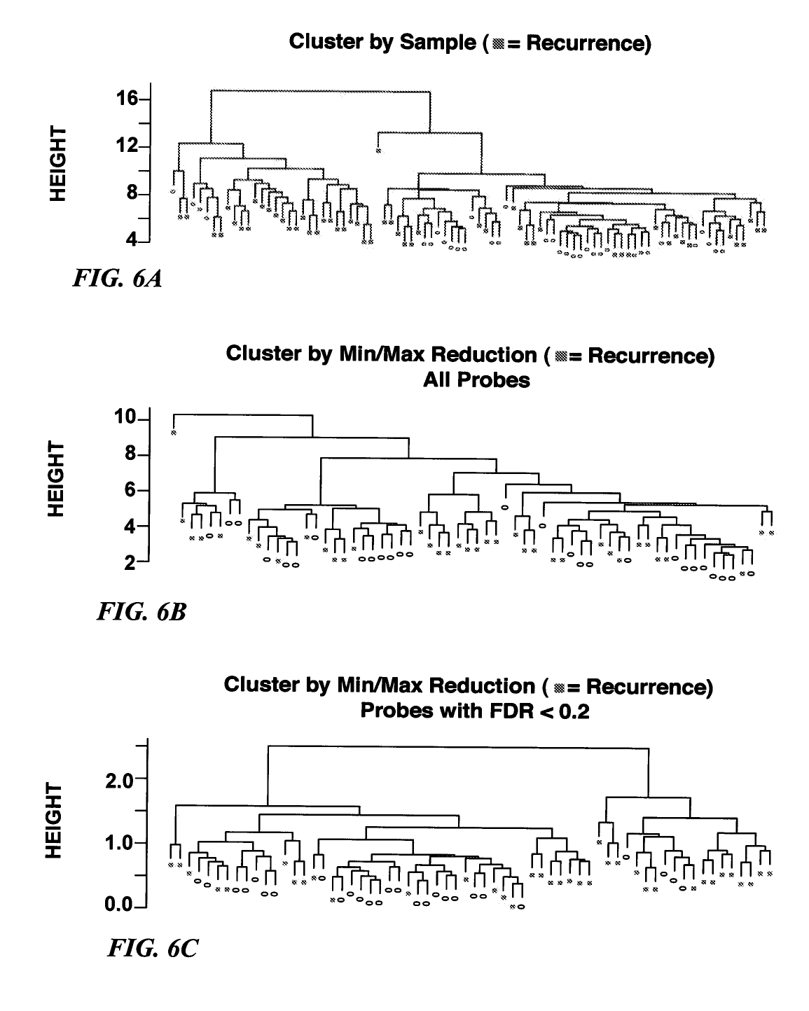

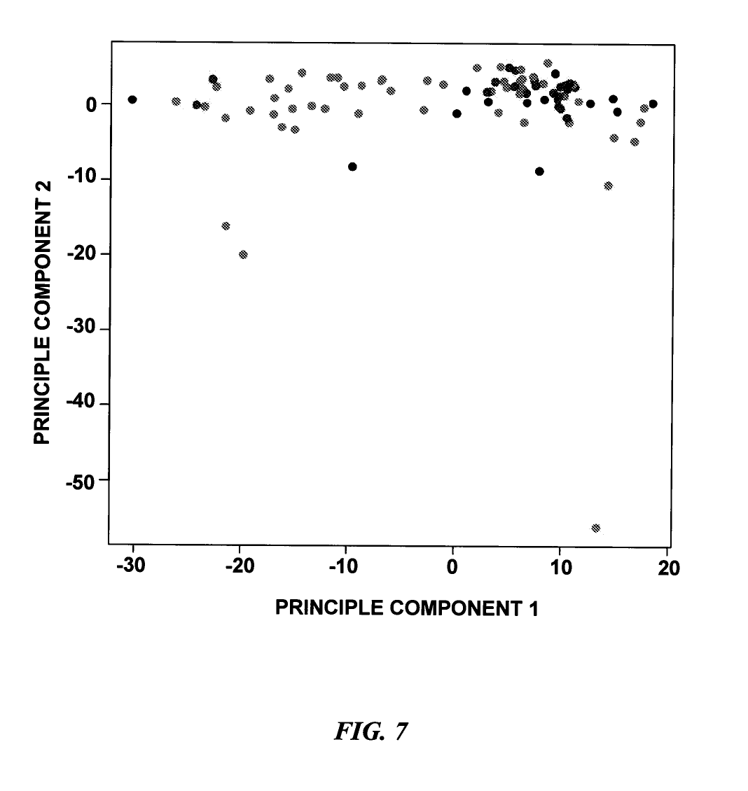

The results of unsupervised hierarchical clustering of all 88 samples (using all 847 miRNA probe sets without MIN-MAX) show that patients with recurrent disease tend to cluster together (FIG. 6A). Employing the MIN-MAX method reduces the results to 64 patients with a very similar clustering, suggesting that information is not grossly distorted or lost as a result of the MIN-MAX procedure (FIG. 6B). When the MIN-MAX method is applied and then a univariate Cox regression analysis is performed, clustering of the patients using probes with FDR<0.2 reveals a clearer distinction between recurrent and nonrecurrent patients (FIG. 6C). To further investigate the clustering between recurrence and nonrecurrence samples, the first two principal components were examined (FIG. 7). As observed in the hierarchical clustering, there is a cluster of primarily recurrence samples and a cluster of mixed recurrence and nonrecurrence.

### Example 4

### HCC Recurrence miRNA Biomarker Discovery and its Comparison to the Milan Criteria

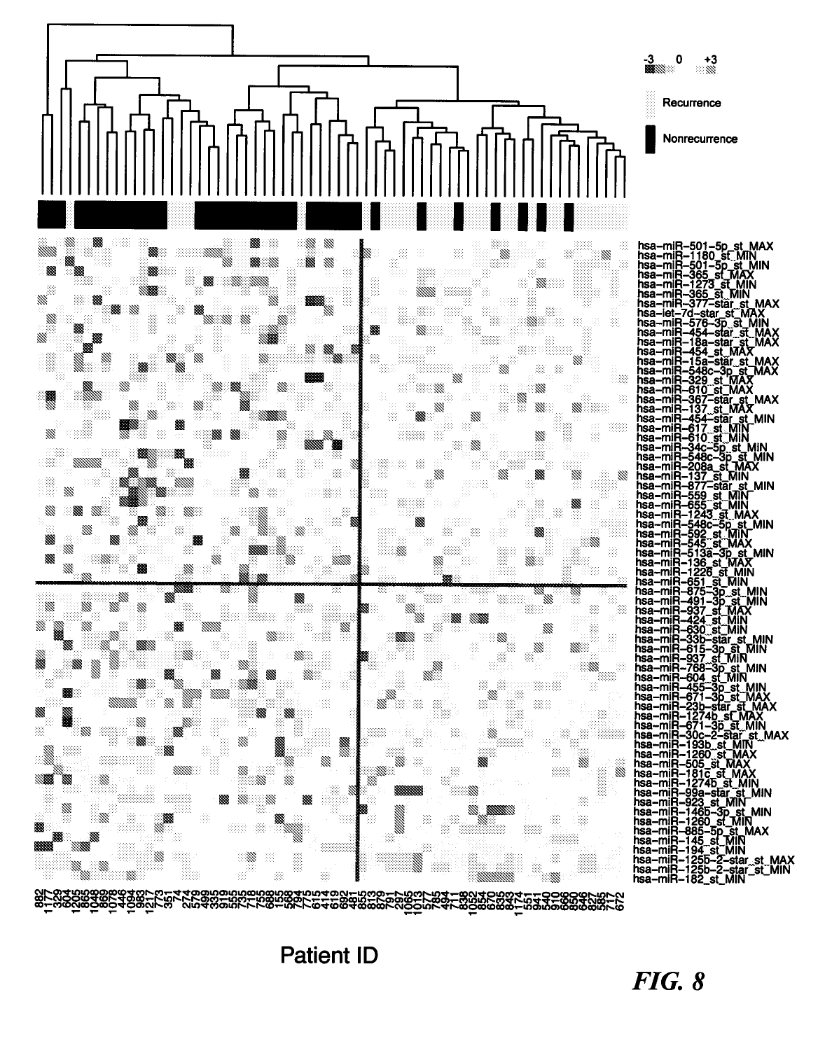

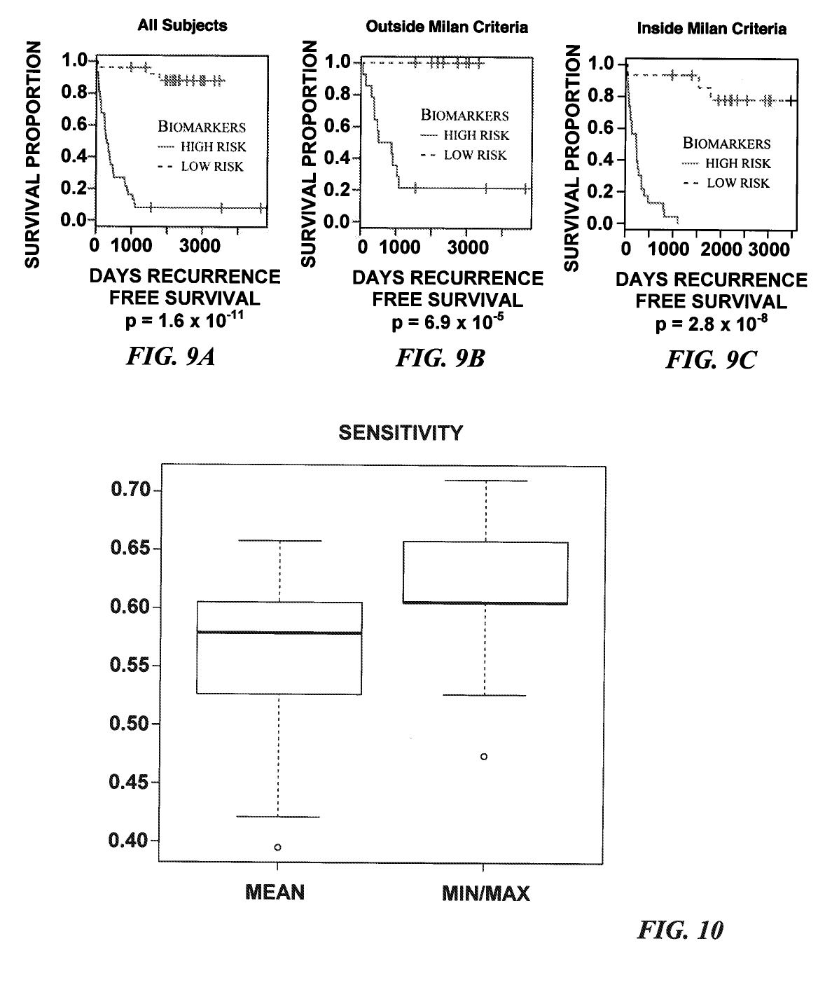

The proposed biomarker was generated using all available data. The biomarker for HCC recurrence utilized 67 miRNAs that significantly distinguished patients with HCC recurrence after transplant from those without recurrence (FIG. 8) with R2 0.848 and AUC=0.989. Analysis of recurrence free survival shows that the biomarker clearly delineates patients with and without recurrence within three years of transplant (FIG. 9A) with a p value of 1.6×10⁻¹¹. Applying the biomarker to patients in the cohort outside Milan (FIG. 9B) and inside Milan (FIG. 9C) also yields statistically significant separation (p=6.9×10⁻⁵). Further, the biomarker can identify patients outside of Milan who have favorable biology and patients within Milan who have unfavorable tumor biology (as measured by disease recurrence). In fact, in this cohort, the biomarker identified 9 of 12 patients within Milan who recurred and 8 of 11 patients outside of Milan who did not recur (Table 3).

TABLE 3

| | 29 Nonrecurrent | 40 Recurrent |
|---|---|---|
| Within Milan (n = 28) | 17 | 11 (8/11 BM) |
| Outside Milan (n = 41) | 12 (9/12 BM) | 29 |

Table 4 lists all probe features appearing in at least 50% of CV biomarkers for the MIN-MAX model with Milan incorporated. The median number of features used in the CV fits was 75 with a minimum of 44 and a maximum of 144. The collinearity plays an important role, in that the fitting procedure tends to exclude features that are highly correlated with features already incorporated in the biomarker. It is interesting to note that the highest ranking feature in the univariate analysis (hsa-miR-194_st_MIN, Table 2) has Pearson's correlation coefficients of 0.58, 0.81 and 0.57 with features hsa-miR-125b-2-star_st_MIN, hsamiR-122_st_MIN and hsa-miR-182_st_MIN, respectively. These are all among the top five ranked features, but the latter t vo appear in less than 50% of CV biomarkers.

TABLE 4

| Probe Feature | Proportion of CV fits |
|---|---|
| hsa-miR-454-star_st_MAX | 1.000 |
| hsa-miR-885-5p_st_MAX | 0.994 |
| hsa-miR-501-5p_st_MIN | 0.992 |
| hsa-miR-454-star_st_MIN | 0.982 |
| hsa-miR-501-5p_st_MAX | 0.978 |
| hsa-miR-365_st_MIN | 0.976 |
| hsa-miR-1273_st_MIN | 0.962 |
| hsa-miR-365_st_MAX | 0.960 |
| hsa-miR-1274b_st_MIN | 0.948 |
| hsa-miR-194_st_MIN | 0.936 |
| hsa-miR-125b-2-star_st_MIN | 0.926 |
| hsa-miR-610_st_MAX | 0.890 |
| hsa-miR-592_st_MIN | 0.850 |
| hsa-miR-137_st_MIN | 0.848 |
| hsa-miR-193b_st_MIN | 0.798 |
| hsa-miR-545_st_MAX | 0.786 |
| hsa-miR-146b-3p_st_MAX | 0.770 |
| hsa-miR-20a-star_st_MIN | 0.768 |
| hsa-miR-1293_st_MAX | 0.756 |
| hsa-miR-424_st_MIN | 0.744 |
| hsa-miR-671-3p_st_MAX | 0.736 |
| hsa-miR-377-star_st_MAX | 0.732 |
| hsa-miR-937_st_MIN | 0.724 |
| hsa-miR-548c-3p_st_MIN | 0.706 |
| hsa-miR-576-3p_st_MIN | 0.704 |
| hsa-miR-146b-3p_st_MIN | 0.700 |
| hsa-miR-513a-3p_st_MIN | 0.692 |
| hsa-miR-137_st_MAX | 0.688 |
| hsa-miR-630_st_MIN | 0.664 |
| hsa-miR-671-3p_st_MIN | 0.644 |
| hsa-miR-1274b_st_MAX | 0.630 |
| hsa-miR-1180_st_MIN | 0.590 |
| hsa-miR-505_st_MAX | 0.578 |
| hsa-miR-424_st_MAX | 0.558 |
| hsa-miR-15a-star_st_MAX | 0.544 |
| hsa-miR-485-3p_st_MAX | 0.538 |
| hsa-miR-182_st_MIN | 0.538 |
| hsa-miR-610_st_MIN | 0.536 |
| hsa-miR-937_st_MAX | 0.526 |
| hsa-miR-1260_st_MIN | 0.524 |
| hsa-miR-1179_st_MIN | 0.524 |
| hsa-miR-99a-star_st_MIN | 0.518 |
| hsa-miR-491-3p_st_MIN | 0.508 |
| hsa-miR-145_st_MIN | 0.504 |

### Example 5

### Biomarker Discovery is Facilitated by the MIN-MAX Method

Perhaps the most common way to handle multifocal data is to compute the average expression across samples for each patient. (This is referred to in the Examples as the “mean” method or procedure.) However, this ignores the possibility of heterogeneity in both the tumor phenotype and expression profile. In this study, it may be reasonable to assume that tumors in nonrecurrent patients are more homogeneous as they all lack the recurrence biomarker. However, recurrence likely only requires the recurrence biomarker to be present in one of a patient's tumors. The result of the CV comparison of the mean and the MIN-MAX procedures support this belief (FIG. 10) as the methods performed comparably with regard to specificity but the MIN-MAX procedure had much better sensitivity.

### Example 6

### Cross Validation Reveals Synergy Between MIN-MAX Biomarker and Milan

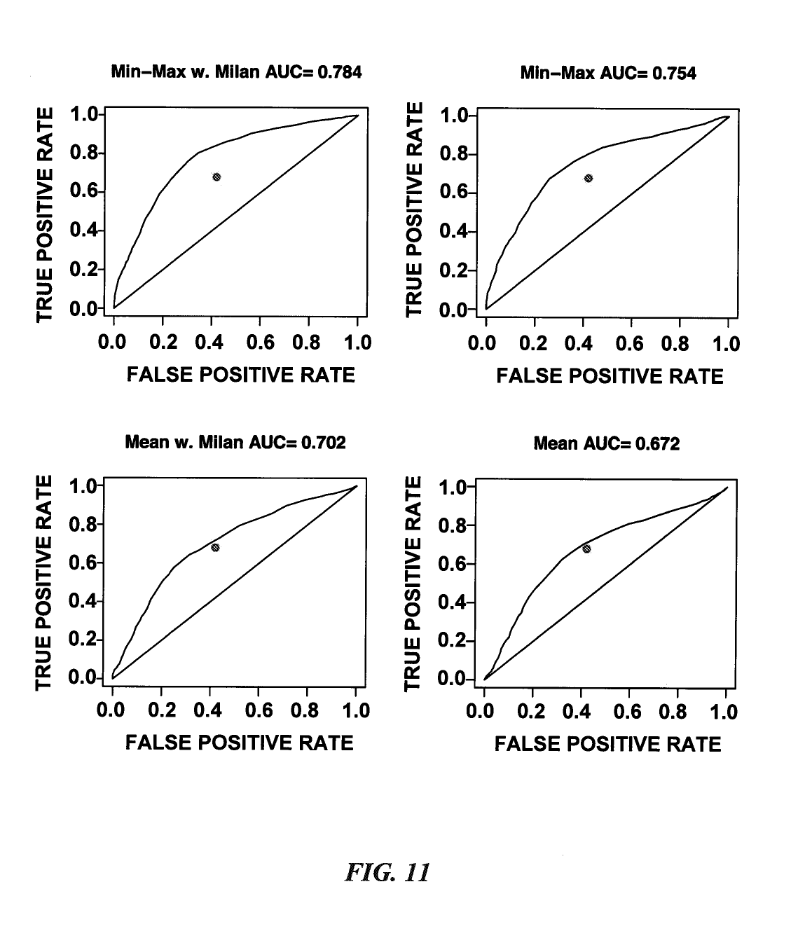

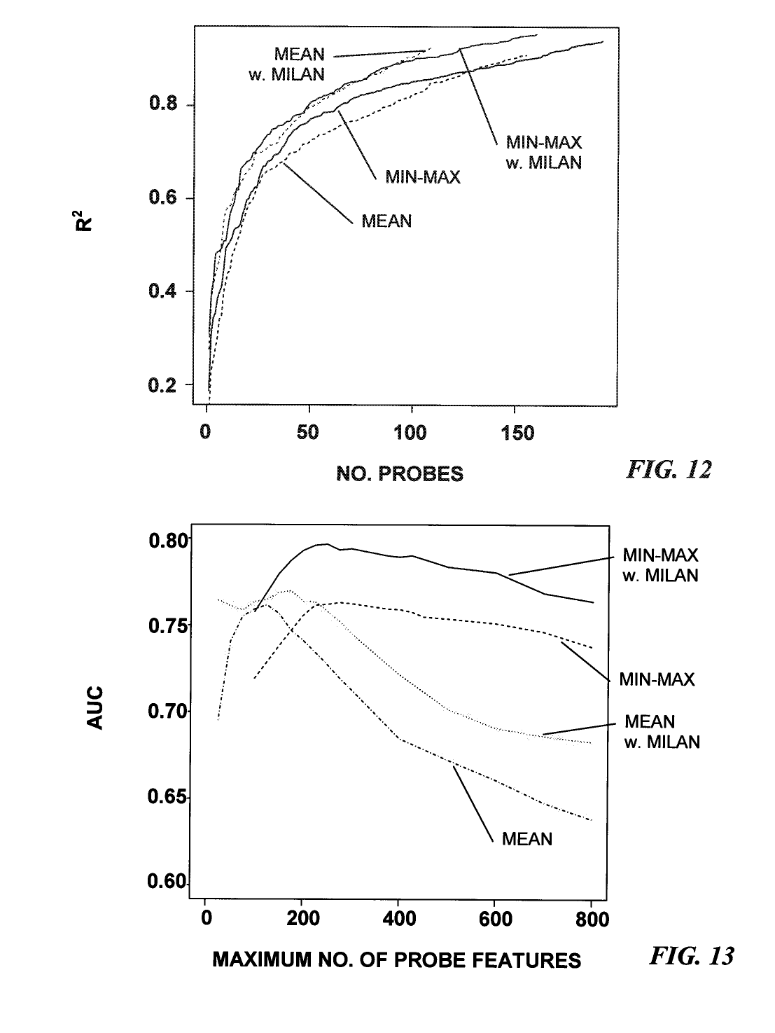

The four receiver operator characteristic plots are shown in FIG. 11. The sensitivity and specificity attained by Milan is superimposed. The MIN-MAX procedure is clearly able to exceed Milan in prognostic accuracy. Furthermore, this accuracy is enhanced by incorporating Milan itself in the biomarker. FIG. 12 demonstrates the construction of the biomarker, indicating the improvement in the R2 value as additional features are added. The rate of increase clearly increases when Milan is included in the model, indicating greater predictive ability of the probes when Milan information is incorporated. Finally, CV was used to assess the optimal number of features to retain in step (2) of the biomarker generation procedure. The resulting AUC statistics are shown in FIG. 13. The prognostic ability of the various markers is clearly sensitive to this parameter, but the superiority of the MIN-MAX biomarker which incorporates Milan is evident over the whole range.

### Discussion of Examples 1-6

There is a growing consensus that evaluation of HCC tumor biology via molecular characterization holds most promise in achieving accurate clinical risk stratification of patients (Zimmerman et al., “Recurrence of Hepatocellular Carcinoma Following Liver Transplantation: A Review of Preoperative and Postoperative Prognostic Indicators,” Arch. Surg. 143:182-188 (2008); Schwartz et al., “Liver Transplantation for Hepatocellular Carcinoma: Are the Milan Criteria Still Valid?” Eur. J. Surg. Oncol. 34:256-262 (2008), which are hereby incorporated by reference in their entirety). Intriguing preliminary results with various biologic metrics such as fractional allelic imbalance (Schwartz et al., “Liver Transplantation for Hepatocellular Carcinoma: Extension of Indications Based on Molecular Markers,” Hepatol. 49:581-588 (2008), which is hereby incorporated by reference in its entirety) and gene expression profiles (Ye et al., “Predicting Hepatitis B Virus-Positive Metastatic Hepatocellular Carcinomas Using Gene Expression Profiling and Supervised Machine Learning,” Nat. Med. 9:416-423 (2003); Lee et al., “Classification and Prediction of Survival in Hepatocellular Carcinoma by Gene Expression Profiling,” Hepatology 40:667-676 (2004); Iizuka et al., “Predicting Individual Outcomes in Hepatocellular Carcinoma,” Lancet 364:1837-1839 (2004); Hoshida et al., “Gene Expression in Fixed Tissues and Outcome in Hepatocellular Carcinoma,” N. Engl. J. Med. 359:1995-2004 (2008), which are hereby incorporated by reference in their entirety) strongly suggest that addition of tumor biology information to current liver transplant selection criteria (i.e., Milan criteria) is possible and desirable. It will soon be reasonable to pursue biomarker testing in pretransplant tumor biopsy specimens to more efficiently direct our resources and improve transplant outcomes, as well as to direct other available therapeutic modalities for HCC.

MicroRNAs are attractive markers as they are known to be master regulators of gene expression and are highly effective in classifying tissue types and tumor tissues of origin (Landgraf et al., “A Mammalian MicroRNA Expression Atlas Based on Small RNA Library Sequencing,” Cell 129:1401-1414 (2007); Lu et al. “MicroRNA Expression Profiles Classify Human Cancers,” Nature 435:834-838 (2005), which are hereby incorporated by reference in their entirety). One potential advantage to studying miRNA over mRNA in biomarker signature building is that there are only just over 1400 miRNAs compared to the over 20000 mRNAs. Therefore, statistical analysis is inherently less noisy and tighter. Also, the small size and stability of miRNAs make them far more amenable to analysis from FFPE tissues compared to much larger and less stable mRNAs.

Evidence already exists that the many miRNAs may be important to hepatocellular carcinogenesis. MiR-194 has been shown to be expressed in hepatic epithelial cells and to suppress HCC metastasis in a murine model (Chen et al., “Gene Expression Patterns in Human Liver Cancers,” Mol. Biol. Cell 13:1929-1939 (2002), which is hereby incorporated by reference in its entirety). This miRNA has also been shown to be downregulated in human HCCs that metastasize (Budhu et al., “Identification of Metastasis-Related MicroRNAs in Hepatocellular Carcinoma,” Hepatology 47:897-907 (2008), which is hereby incorporated by reference in its entirety). MiR-125b-2* is expressed in human fetal liver cells (Budhu et al., “Prediction of Venous Metastases, Recurrence, and Prognosis in Hepatocellular Carcinoma Based on a Unique Immune Response Signature of the Liver Microenvironment,” Cancer Cell 10:99-111 (2006), which is hereby incorporated by reference in its entirety) and its dysregulated expression is noted in colorectal cancer with liver metastases (Dobbin et al., “How Large a Training Set is Needed to Develop a Classifier for Microarray Data?” Clin. Cancer Res. 14 108-114 (2008), which is hereby incorporated by reference in its entirety). MiR-182 expression has been shown in two independent studies comparing HCC to adjacent uninvolved liver to be significantly upregulated (Sato et al., “MicroRNA Profile Predicts Recurrence After Resection in Patients With Hepatocellular Carcinoma Within the Milan Criteria,” PLoS One 6:e16435 (2011); Wang et al., “Profiling MicroRNA Expression in Hepatocellular Carcinoma Reveals MicroRNA-224 Up-Regulation and Apoptosis Inhibitor-5 as a MicroRNA-224-Specific Target,” J. Biol. Chem. 283:13205-13215 (2008), which are hereby incorporated by reference in their entirety). All HCC recurrence miRNA studies to date have been performed in the context of hepatic resection rather than transplant. It is expected, therefore, that the Min-Max procedure can be used to define a risk score for predicting recurrence in hepatic resection patients as well.

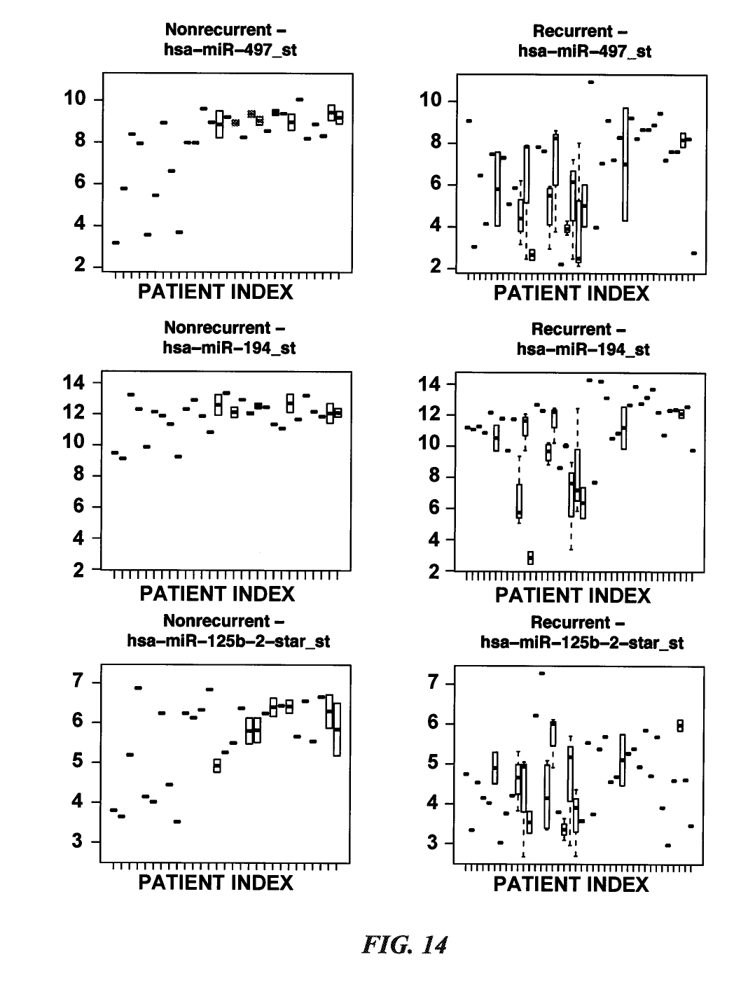

When the variation in expression of particular miRNAs is examined in the context of individual patients, there is more probe expression variation in recurrent patients versus nonrecurrent (FIG. 14). In fact, of the 847 miRNAs, over 85% show greater average within-patient variance in the recurrence group than in the nonrecurrence group. This is strongly suggestive of the distributional mixture, which would result from nonhomogeneity of genetic response among multifocal samples. This argues strongly that the MIN-MAX approach should be used to select which of the varied expression levels for a given miRNA is driving the phenotype.

Previously published gene expression profiling studies of HCC using microarrays to assay mRNA changes have not sufficiently addressed the multifocal issue (Ye et al., “Predicting Hepatitis B Virus-Positive Metastatic Hepatocellular Carcinomas Using Gene Expression Profiling and Supervised Machine Learning,” Nat. Med. 9; 416-423 (2003); Lee et al., “Classification and Prediction of Survival in Hepatocellular Carcinoma by Gene Expression Profiling,” Hepatology 40:667-676 (2004); Iizuka et al., “Predicting Individual Outcomes in Hepatocellular Carcinoma,” Lancet 364:1837-1839 (2004); Hoshida et al., “Gene Expression in Fixed Tissues and Outcome in Hepatocellular Carcinoma,” N. Engl. J. Med. 359:1995-2004 (2008); Budhu et al., “Identification of Metastasis-Related MicroRNAs in Hepatocellular Carcinoma,” Hepatology 47:897-907 (2008); Sato et al., “MicroRNA Profile Predicts Recurrence After Resection in Patients With Hepatocellular Carcinoma Within the Milan Criteria,” PLoS One 6:e16435 (2011); Toffanin et al., “MicroRNA-Based Classification of Hepatocellular Carcinoma and Oncogenic Role of miR-517a,” Gastroenterology 140:1618-1628 (2011), which are hereby incorporated by reference in their entirety). The rationale for this is based on the observation that metastatic tumors from an individual patient tend to have gene expression profiles that are far more similar to that patient's primary tumor compared to expression profiles from other patients' tumors (Chen et al., “Gene Expression Patterns in Human Liver Cancers,” Mol. Biol. Cell 13:1929-1939 (2002); Budhu et al., “Prediction of Venous Metastases, Recurrence, and Prognosis in Hepatocellular Carcinoma Based on a Unique Immune Response Signature of the Liver Microenvironment,” Cancer Cell 10:99-111 (2006), which are hereby incorporated by reference in their entirety). The current analysis, however, demonstrates that miRNA expression profiles can vary significantly between multifocal tumors from the same patient and this is therefore likely the case with other forms of RNA, including mRNA. Application of the MIN-MAX procedure to mRNA signature discovery in HCC may significantly reduce false discovery of genes thought to be associated with important clinical outcomes such as survival, metastasis, and recurrence. The MIN-MAX approach is generalizable and can be applied to other multifocal disease processes such as the analysis of metastatic lesions or premalignant lesions in HCC and other cancer types.

The cohort of this study is somewhat unique in that there was a high degree of HCC recurrence. This can be attributed to a previously aggressive practice of transplanting patients who were outside of Milan criteria, because the overall recurrence rate of patients transplanted within Milan is 10.5% over this 12-year period. The only change in the immunosuppressive management over this period was the introduction of mycophenolate mofetil in 2001 and an era effect on recurrence as a result of this change was not detected.

Global miRNA analysis of FFPE samples from explanted HCCs can be used to develop molecular signatures defining clinically important outcomes and presented herein is a miRNA biomarker that distinguishes patients with and without recurrent HCC within 3 years of transplant. The MIN-MAX method is effective in directing appropriate probe selection when analyzing multifocal specimens. This biologic metric can be used in concert with the existing Milan criteria to more efficiently utilize resources and improve outcomes in liver transplant for patients with HCC. The biomarker may also be used to help rationally direct other HCC treatments such as chemotherapy, ablation, and resection.

### Example 7

### Tissue-Based Vs. Subject-Based Biomarkers

The advantage of multiple sample aggregation is demonstrated in the following comparison. The Standardized Scale Model described above may be applied directly to expression profiles for each tissue (Min/Max aggregation is therefore not required in this case). This yields a recurrence prediction, or risk score, for each tissue, and therefore multiple predictions for any subject with multiple tissue samples. For any one subject, this in turn leads to the potential for conflicting predictions.

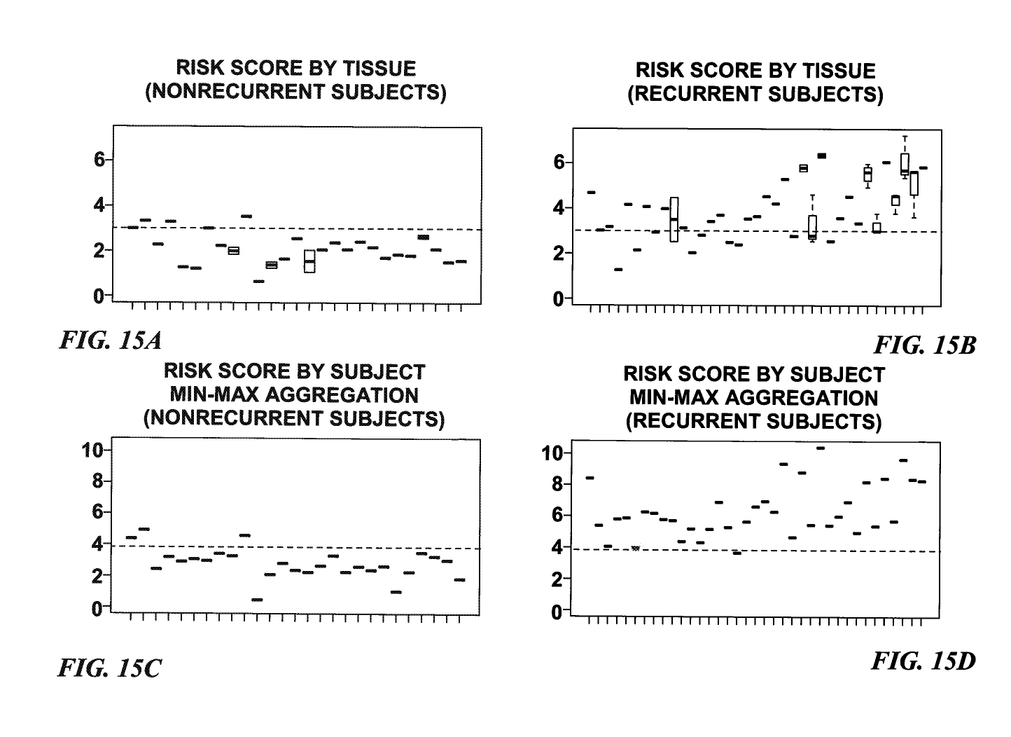

FIG. 15 displays the risk scores obtained by applying the SSM biomarker methodology directly to the tissue expression profiles (FIGS. 15A-15B) and to the subject level Min/Max aggregations as described in the methodology (FIGS. 15C-15D). The risk scores are displayed by subject, and tissue-specific risk scores associated with subjects with multiple samples are summarized by boxplots. Risk scores are further divided into separate plots for recurrent (FIGS. 15B and 15D) and non-recurrent subjects (FIGS. 15A and 15C).

For each biomarker, the 90^{th} percentile of the risk scores for non-recurrent subjects is shown in FIGS. 15A and 15C as a dashed line (3.0 for tissue-based, 3.8 for subject-based). This estimates a risk threshold with 90% specificity. The overall viability of either biomarker as a prognostic score can be clearly seen by the preponderance of risk scores above the 90% threshold among the recurrent subjects. However, the subject-based risk score (FIG. 15D) has significantly higher sensitivity than the tissue-based risk score (FIG. 15B) (97% vs 72%), interpretable as the proportion of recurrent subjects (or of tissue samples from recurrent subjects) with risk scores higher than the respective 90% threshold. It can also be seen in FIG. 15B that in some cases, recurrent subjects with multiple tissue samples possess tissue-based risk scores with conflicting predictions, that is, with risk scores both above and below the 90% threshold. Therefore, the Min/Max aggregation method described supra is demonstrated to result in greater prognostic accuracy, and is able to resolve potentially conflicting risk predictions that would result from the use of tissue-specific biomarkers.

This example illustrates the intended use of the HCC biomarker. The SSM methodology yields a biomarker that produces a quantitative reference threshold score that is oriented so that high scores are predictive of recurrence. The biomarker development process will yield separate and observable sample distributions of risk scores for both the recurrent and non-recurrent phenotype. In the simplest mode of use, a threshold is defined (at a certain specificity/sensitivity) yielding a prediction of recurrence when the risk score exceeds this value. In practice, selection of this threshold may depend on several criteria. Ideally, a threshold perfectly separates recurrent and non-recurrent risk scores, but in practice a threshold is selected to yield a satisfactory balance of false positives (non-recurrent subjects with risk scores above the threshold) and false negatives (recurrent subjects with risk scores below the threshold). Determination of this threshold will be based on established clinical and epidemiological methodologies, and may incorporate information such as donor availability or auxiliary subject risk factors.

### Example 8

### Application of Min-Max Method to Larger HCC Cohort

While the relatively small number of patients studied in Examples 1-7 was sufficient to demonstrate efficacy of the Min-Max procedure, expansion of the test cohort to several hundred patients where examination of all tumor nodules in every patient is performed, will be used to further assess the performance of the MIN-MAX procedure and to refine the miRNA biomarker. Quantitative PCR is being used to verify the 67-miRNA biomarker and to complete signature building on an additional 150 tumors from 50 patients. In addition, another 150 tumors from 50 more patients will be used to perform external CV. While the external validation will require fewer patients (Dobbin et al., “How Large a Training Set is Needed to Develop a Classifier for Microarray Data?” Clin. Cancer Res. 14:108-114 (2008), which is hereby incorporated by reference in its entirety), complete surveillance of each individual's entire tumor burden is expected to clearly define which miRNAs are truly driving the clinical phenotype of interest. Because the 67-miRNA biomarker from Examples 1-7 outperformed other criteria (Milan and Mean procedure), it is expected that expansion of this study will result in a more comprehensive and clinically robust miRNA biomarker for HCC.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of determining a subject's risk of developing recurrent hepatocellular carcinoma comprising:
contacting an isolated hepatocellular carcinoma sample from the subject with reagents suitable for detecting expression levels of two or more of microRNAs in the sample;
measuring the expression levels of the two or more microRNAs in the sample based on said contacting;
calculating a risk score of hepatocellular carcinoma disease recurrence for the subject based on the measured microRNA expression levels in the isolated sample; and
comparing the calculated risk score for the subject to a reference score of hepatocellular carcinoma disease recurrence to determine the subject's risk of developing recurrent hepatocellular carcinoma.

2. The method of claim 1, wherein the two or more microRNAs are selected from the group of microRNAs consisting of hsa-miR-501, hsa-miR-1180, hsa-miR-365, hsa-miR-1273, hsa-miR-377, hsa-let-7d, hsa-miR-576, hsa-miR-454, hsa-miR-18a, hsa-miR-15a, hsa-miR-548c, hsa-miR-20a, hsa-miR-610, miR-146b, hsa-miR-137, hsa-miR-1293, hsa-miR-139, hsa-miR26a, hsa-miR-122, hsa-miR-192, hsa-miR-888, hsa-miR-497, hsa-miR-592, hsa-miR-545, hsa-miR-513a, hsa-miR-136, hsa-miR-1226, hsa-miR-651, hsa-miR-542, hsa-miR-491, hsa-miR-937, hsa-miR-424, hsa-miR-630, hsa-miR-33b, hsa-miR-615, hsa-mir-152, hsa-miR-455, hsa-miR-23b, hsa-miR-671, hsa-miR-30c-2, hsa-miR-193b, hsa-miR-1260, hsa-miR-505, hsa-miR-181c, hsa-miR-99a, hsa-miR-885, hsa-miR-145, hsa-miR-194, hsa-miR-125b-2, hsa-miR-182

3. The method of claim 1, wherein the two or more microRNAs are selected from the group consisting of hsa-miR-454, hsa-miR-885, hsa-miR-365, hsa-miR-501, hsa-miR-194, hsa-miR-125b-2, hsa-miR-20a, hsa-miR-146b, hsa-miR-137, hsa-miR-1273, hsa-miR-424, hsa-miR-610, hsa-miR-1293, hsa-miR-505, hsa-miR-377, hsa-miR-1260, hsa-miR-182, hsa-miR-1180, hsa-miR-592, hsa-miR-576, hsa-miR-630, hsa-miR-99a, hsa-let-7d, hsa-miR-139, hsa-miR-26a-2, hsa-miR-193b, hsa-miR-122, hsa-miR-192, hsa-miR-885, hsa-miR-888, hsa-miR-497, hsa-miR-542, and hsa-miR-152.

4. The method of claim 1, wherein when the subject has multiple tumor lesions, said method further comprising:
isolating a hepatocellular carcinoma sample from more than one of the multiple tumor lesions in the subject and
measuring minimum and maximum expression levels of the two or more microRNAs in each of the isolated samples, wherein the risk score for the subject is calculated based on both of the measured minimum and maximum microRNA expression levels, if different, in the isolated samples.

5. The method of claim 1 wherein said calculating a risk score of hepatocellular carcinoma disease recurrence comprises:
standardizing the measured expression level of each of the two or more microRNAs to a reference distribution expression value for each microRNA;
calculating the sum of the standardized microRNAs expression levels from the subject.

6. The method of claim 1, wherein said standardizing the measured expression level is carried out according to the equation of formula (I):
Zi=(i−i_{median})/i_{IQR} (I)
where
Zi is the standardized expression value
i is the minimum or maximum measured expression level of the microRNA in an individual of a cohort of recurrent and non-recurrent hepatocellular carcinoma patient
i_{median} is the median expression level of the microRNA calculated across the cohort of patients
i_{IQR} is the interquartile range of expression level of the corresponding molecular biomarker calculated across the cohort of patients; and.
wherein
when Zi is <0, Zi is multiplied by −1.

7. The method of claim 1, wherein when the calculated risk score for the subject is greater than the reference threshold score for hepatocellular carcinoma disease recurrence, the subject has a high risk of developing recurrent hepatocellular carcinoma.

8. The method of claim 1, wherein when the calculated risk score for the subject is lower than the reference threshold score for hepatocellular carcinoma disease recurrence, the subject has a low risk of developing recurrent hepatocellular carcinoma.

9. The method of claim 1 further comprising:
evaluating one or more additional prognostic criteria and
determining the subject's risk of developing recurrent hepatocellular carcinoma based on the combination of said comparing and said evaluating.

10. The method of claim 9, wherein the one or more additional prognostic criteria comprise cancer lesion size, the number of cancerous lesions, the presence of extrahepatic manifestations, vascular invasion, and any combination thereof.

11. The method of claim 1 further comprising:
administering a suitable therapy to said subject based on the determined risk.

12. A method of treating a subject having hepatocellular carcinoma comprising:
calculating the subject's risk score of hepatocellular carcinoma disease recurrence based on measured expression levels of two or more microRNAs in one or more isolated hepatocellular carcinoma samples from the subject;
comparing the calculated risk score for the subject to a reference threshold score of hepatocellular carcinoma disease recurrence; and
administering a suitable therapy for said subject based on the calculated risk score of hepatocellular carcinoma disease recurrence.

13. The method of claim 12, wherein said calculating comprises:
contacting the one or more isolated hepatocellular carcinoma samples from the subject with reagents suitable for detecting the expression levels of the two or more microRNAs in each of the one or more samples;
measuring the minimum and maximum expression levels of the two or more microRNAs in the one or more samples based on said contacting;
standardizing the measured minimum and maximum expression levels of each of the two or more microRNAs to a reference distribution expression value; and
calculating the sum of the standardized microRNAs expression levels from the subject.

14. The method of claim 12, wherein a suitable therapy for a subject having a higher calculated risk score than the reference threshold score for hepatocellular carcinoma disease recurrence comprises one or more therapies selected from the group consisting of transcatheter arterial chemoembolization, radiofrequency ablation, surgical resection, radiotherapy, or chemotherapy.

15. The method of claim of claim 12, wherein a suitable therapy for a subject having a lower calculated risk score than the reference threshold score for heptocellular carcinoma disease recurrence comprises liver transplantation.

16. A kit comprising:
a collection of oligonucleotides, said collection consisting essentially of two or more oligonucleotides that hybridize under stringent conditions to two or more microRNAs, respectively, wherein the two more microRNAs are selected from the group of hsa-miR-501, hsa-miR-1180, hsa-miR-365, hsa-miR-1273, hsa-miR-377, hsa-let-7d, hsa-miR-576, hsa-miR-454, hsa-miR-18a, hsa-miR-15a, hsa-miR-548c, hsa-miR-20a, hsa-miR-610, miR-146b, hsa-miR-137, hsa-miR-1293, hsa-miR-139, hsa-miR26a, hsa-miR-122, hsa-miR-192, hsa-miR-888, hsa-miR-497, hsa-miR-592, hsa-miR-545, hsa-miR-513a, hsa-miR-136, hsa-miR-1226, hsa-miR-651, hsa-miR-542, hsa-miR-491, hsa-miR-937, hsa-miR-424, hsa-miR-630, hsa-miR-33b, hsa-miR-615, hsa-mir-152, hsa-miR-455, hsa-miR-23b, hsa-miR-671, hsa-miR-30c-2, hsa-miR-193b, hsa-miR-1260, hsa-miR-505, hsa-miR-181c, hsa-miR-99a, hsa-miR-885, hsa-miR-145, hsa-miR-194, hsa-miR-125b-2, hsa-miR-182

17. The kit of claim 16, wherein the two more microRNAs are selected from the group of hsa-miR-454, hsa-miR-885, hsa-miR-365, hsa-miR-501, hsa-miR-194, hsa-miR-125b-2, hsa-miR-20a, hsa-miR-146b, hsa-miR-137, hsa-miR-1273, hsa-miR-424, hsa-miR-610, hsa-miR-1293, hsa-miR-505, hsa-miR-377, hsa-miR-1260, hsa-miR-182, hsa-miR-1180, hsa-miR-592, hsa-miR-576, hsa-miR-630, hsa-miR-99a, hsa-let-7d, hsa-miR-139, hsa-miR-26a-2, hsa-miR-193b, hsa-miR-122, hsa-miR-192, hsa-miR-885, hsa-miR-888, hsa-miR-497, hsa-miR-542, and hsa-miR-152.

18. The kit of claim 16, wherein one or more of the oligonucleotides in the collection comprise a detectable label.

19. The kit of claim 16 further comprising one or more reagents selected from the group consisting of reverse transcriptase, polymerase, dNTPs, and one or more buffer solutions.

20. The kit of claim 16 further comprising:
instructions for using the collection of oligonucleotides to measure microRNA expression levels and
a computer readable medium having stored thereon instructions for defining a risk score hepatocellular carcinoma disease recurrence based on said measured microRNA expression levels.

21. A method of defining a biomarker reference threshold score that correlates with a disease phenotype, said method comprising:
obtaining one or more disease samples from each individual in a cohort of patients having different disease phenotypes;
contacting the one or more obtained disease samples with reagents suitable for detecting expression levels of two or more candidate molecular biomarkers;
measuring the minimum and maximum expression levels of the candidate molecular biomarkers within the one or more disease samples from each individual based on said contacting;
selecting the minimum and/or maximum expression levels of the candidate molecular biomarkers that significantly correlate with a disease phenotype of the patient cohort;
generating a standardized expression value for each of the selected minimum and maximum expression levels;
constructing a biomarker reference score for each individual in the cohort by summing the standardized expression values of said candidate molecular biomarkers whose inclusion in the sum maximizes the correlation between the biomarker reference score and the disease phenotype; and
summarizing biomarker reference score distribution across the cohort to define a biomarker reference threshold score.

22. The method of claim 21, wherein said selecting further comprises:
assessing a statistical association of the minimum and maximum expression levels of each candidate molecular biomarker with a disease phenotype;
assigning p-values to the identified minimum and maximum expression levels; and
ranking, in order of most to least significant p-values, the identified minimum and maximum expression levels.

23. The method of claim 21, wherein said generating a standardized expression value is carried out according to the equation of formula (I):
Zi=(i−i_{median})/i_{IQR} (I)
where
Zi is the standardized expression value
i is the minimum or maximum measured expression level of the molecular biomarker in an individual of the cohort of patients
i_{median} is the median expression level of the molecular biomarker calculated across the cohort of patients
i_{IQR} is the interquartile range of expression level of the molecular biomarker calculated across the cohort of patients; and.
wherein
when Zi is <0, Zi is multiplied by −1.

24. The method of claim 21, wherein when two or more different disease samples are obtained from an individual of the cohort and said measured minimum and/or maximum expression values of a candidate molecular biomarker are different in said two more samples, said selecting comprises:
selecting the lowest minimum expression level and the highest maximum expression level from the two or more samples that correlate with a disease phenotype of the patient cohort.

25. The method of claim 21, wherein the candidate molecule biomarkers are selected from the group consisting of mRNA expression levels, microRNA expression levels, protein expression levels, and metabolite concentrations.

26. The method of claim 25, wherein when said molecular biomarkers comprise mRNAs or microRNA expression levels, said measuring comprises:
measuring, in a hybridization assay, hybridization of one or more oligonucleotide probes comprising a nucleotide sequence that is complementary to at least a portion of a nucleotide sequence of a nucleic acid molecule comprising the molecular biomarker.

27. The method of claim 25, wherein when said candidate molecular biomarkers mRNAs or microRNA expression levels, said measuring comprises:
measuring biomarker amplicon production in an amplification-based assay.

28. The method of claim 25, wherein when said candidate molecular biomarkers comprises protein expression levels, said measuring comprises:
measuring protein expression level using an immunoassay or mass spectroscopy.

29. The method of claim 25, wherein when said candidate molecular biomarkers comprise metabolite concentrations, said measuring comprises:
measuring metabolite concentration using an immunoassay or mass spectroscopy.

30. A method of defining a biomarker reference threshold score that correlates with a disease phenotype, the method comprising:
obtaining from at least one or more sources, by a statistical computing device, minimum and maximum expression levels of candidate molecular biomarkers in one or more disease samples from each individual in a cohort of patients having different disease phenotypes;
selecting, by the statistical computing device, the minimum and/or maximum expression levels of the candidate molecular biomarkers that significantly correlate with a disease phenotype of the patient cohort;
generating, by the statistical computing devise, a standardized expression value for each of the selected minimum and maximum expression levels of the molecular biomarkers; and
constructing, by the statistical computing device, a biomarker reference score for each individual in the cohort by summing the standardized expression values of said candidate molecular biomarkers whose inclusion in the sum maximizes the correlation between the biomarker reference score and the disease phenotype; and
summarizing, by the statistical computing device, biomarker reference score distribution across the cohort to define a biomarker reference threshold score.

31.-40. (canceled)

41. A non-transitory computer readable medium having stored thereon instructions for defining a biomarker reference threshold score that correlates with a disease phenotype comprising machine executable code which when executed by at least one processor, causes the processor to perform steps comprising:
obtaining minimum and maximum expression levels of candidate molecular biomarkers within one or more disease samples from each individual in a cohort of patients having different disease phenotypes;
selecting the minimum and/or maximum expression levels of the candidate molecular biomarkers that significantly correlate with a disease phenotype in the patient cohort;
generating a standardized expression value for each of the selected minimum and maximum expression levels of the molecular biomarkers;
constructing a biomarker reference score for each individual in the cohort by summing the standardized expression values of said candidate molecular biomarkers whose inclusion in the sum maximizes the correlation between the biomarker reference score and the disease phenotype; and
summarizing biomarker reference score distribution across the cohort to define a biomarker reference threshold score.

42.-51. (canceled)

52. A computing device to define a biomarker reference threshold score that correlates with a disease phenotype, the device comprising:
one or more processors and
a memory device coupled to the one or more processors, wherein the one or more processors is configured to execute programmed instructions stored in the memory device comprising:
obtaining minimum and maximum expression levels of candidate molecular biomarkers in one or more disease samples from individuals in a cohort of patients having different disease phenotypes;
selecting the minimum and/or maximum expression levels of the candidate molecular biomarkers that significantly correlate with a disease phenotype in the patient cohort;
generating a standardized expression value for each of the selected minimum and maximum expression levels of the molecular biomarkers;
constructing a biomarker reference score for each individual in the cohort by summing the standardized expression values of said candidate molecular biomarkers whose inclusion in the sum maximizes the correlation between the biomarker reference score and the disease phenotype; and
summarizing biomarker reference score distribution across the cohort to define a biomarker reference threshold score.

53.-62. (canceled)


SEQUENCE LISTING (65 sequences)

SEQ ID NO: 1 - RNA, 115 nt, Homo sapien
ucuguuuaucaccagauccuagaacccuaucaauauugucucugcuguguaaauaguucugaguagugcaauauugcuuauaggguuuugguguuuggaaagaacaaugggcagg

SEQ ID NO: 2 - RNA, 74 nt, Homo sapien
ccgcacucucuccauuacacuacccugccucuucuccaugagaggcagcgggguguaguggauagagcacgggu

SEQ ID NO: 3 - RNA, 87 nt, Homo sapien
accgcagggaaaaugagggacuuuugggggcagauguguuuccauuccacuaucauaaugccccuaaaaauccuuauugcucuugca

SEQ ID NO: 4 - RNA, 111 nt, Homo sapien
agaguguucaaggacagcaagaaaaaugagggacuuucaggggcagcuguguuuucugacucagucauaaugccccuaaaaauccuuauuguucuugcagugugcaucggg

SEQ ID NO: 5 - RNA, 84 nt, Homo sapien
gcucuuccucucuaauccuuugucccugggugagagugcuuucugaaugcaaugcacccgggcaaggauucugagagggugagc

SEQ ID NO: 6 - RNA, 85 nt, Homo sapien
augguguuaucaaguguaacagcaacuccauguggacuguguaccaauuuccaguggagaugcuguuacuuuugaugguuaccaa

SEQ ID NO: 7 - RNA, 85 nt, Homo sapien
ugguucccgcccccuguaacagcaacuccauguggaagugcccacugguuccaguggggcugcuguuaucuggggcgagggccag

SEQ ID NO: 8 - RNA, 89 nt, Homo sapien
accagacuuuuccuagucccugagacccuaacuugugagguauuuuaguaacaucacaagucaggcucuugggaccuaggcggagggga

SEQ ID NO: 9 - RNA, 71 nt, Homo sapien
guagcacuaaagugcuuauagugcagguaguguuuaguuaucuacugcauuaugagcacuuaaaguacugc

SEQ ID NO: 10 - RNA, 73 nt, Homo sapien
ccuggcacugagaacugaauuccauaggcugugagcucuagcaaugcccuguggacucaguucuggugcccgg

SEQ ID NO: 11 - RNA, 102 nt, Homo sapien
gguccucugacucucuucggugacggguauucuuggguggauaauacggauuacguuguuauugcuuaagaauacgcguagucgaggagaguaccagcggca

SEQ ID NO: 12 - RNA, 103 nt, Homo sapien
ugaggcaggagaauugcuugaacccgggugguggagguugcagugagccaagauugcgccacugcacuccagccugggcgacaaagcaagacucuuucuugga

SEQ ID NO: 13 - RNA, 77 nt, Homo sapien
ugcagccugggcgacaaaacgagacccugucuuuuuuuuuuucugagacagagucucguucuguugcccaagcugga

SEQ ID NO: 14 - RNA, 86 nt, Homo sapien
gaaucgcuugaacccaugagguugaggcugcagugagccaagaucgugccacugcacuucagccugggugacaagagcgaaacuuc

SEQ ID NO: 15 - RNA, 102 nt, Homo sapien
ugaggcaggagaauugcuugaacccaggaaguggaggcugcagugagccgagaucgagccacuguacuccagccugggugacacagcgagacuccagucuca

SEQ ID NO: 16 - RNA, 99 nt, Homo sapien
aggugggaggauugcuugagccugggagauggagguugcagugagcugagaucacgcaacugcacccccagccugggccauagagucaguccuugucuc

SEQ ID NO: 17 - RNA, 100 nt, Homo sapien
gaggugggaggauugcuugagucagggugguugaggcugcaguaaguugugaucauaccacugcacuccagccugagugacagagcaagaccuugucuca

SEQ ID NO: 18 - RNA, 98 nt, Homo sapien
cgaggggauacagcagcaauucauguuuugaaguguucuaaaugguucaaaacgugaggcgcugcuauacccccucguggggaagguagaaggugggg

SEQ ID NO: 19 - RNA, 96 nt, Homo sapien
ucuauuugucuuaggugagcuaaaugugugcugggacacauuugagccaaaugucccagcacacauuuagcucacauaagaaaaauggacucuagu

SEQ ID NO: 20 - RNA, 71 nt, Homo sapien
agguuguucuggguggucuggagauuugugcagcuuguaccugcacaaaucuccggaccacuuagucuuua

SEQ ID NO: 21 - RNA, 84 nt, Homo sapien
gaugcacccagugggggagccaggaaguauugauguuucugccaguuuagcgucaacacuugcugguuuccucucuggagcauc

SEQ ID NO: 22 - RNA, 69 nt, Homo sapien
uugagcagagguugcccuuggugaauucgcuuuauuuauguugaaucacacaaaggcaacuuuuguuug

SEQ ID NO: 23 - RNA, 73 nt, Homo sapien
accuuuccagcucaucccaccucugccaccaaaacacucaucgcggggucagagggagugccaaaaaagguaa

SEQ ID NO: 24 - RNA, 89 nt, Homo sapien
ucuccguuuaucccaccacugccaccauuauugcuacuguucagcaggugcugcugguggugauggugauagucuggugggggcggugg

SEQ ID NO: 25 - RNA, 110 nt, Homo sapien
gagcugcuugccuccccccguuuuuggcaaugguagaacucacacuggugagguaacaggauccggugguucuagacuugccaacuauggggcgaggacucagccggcac

SEQ ID NO: 26 - RNA, 69 nt, Homo sapien
gcugcuggacccacccggccgggaauagugcuccugguuguuuccggcucgcgugggugugucggcggc

SEQ ID NO: 27 - RNA, 97 nt, Homo sapien
uauuaugccaugacauugugucaauaugcgaugauguguugugauggcacagcgucaucacguggugacgcaacaucaugacguaagacgucacaac

SEQ ID NO: 28 - RNA, 98 nt, Homo sapien
uacaauccaacgaggauucuaauuucuccacgucuuugguaauaagguuuggcaaagauguggaaaaauuggaauccucauucgauugguuauaacca

SEQ ID NO: 29 - RNA, 97 nt, Homo sapien
aacuuaacaucaugcuaccucuuuguaucauauuuuguuauucuggucacagaaugaccuaguauucuguaccagggaagguaguucuuaacuauau

SEQ ID NO: 30 - RNA, 81 nt, Homo sapien
cccauuggcauaaacccguagauccgaucuuguggugaaguggaccgcacaagcucgcuucuaugggucugugucagugug

SEQ ID NO: 31 - RNA, 87 nt, Homo sapien
ccuaggaagagguaguagguugcauaguuuuagggcagggauuuugcccacaaggagguaacuauacgaccugcugccuuucuuagg

SEQ ID NO: 32 - RNA, 68 nt, Homo sapien
guguauucuacagugcacgugucuccaguguggcucggaggcuggagacgcggcccuguuggaguaac

SEQ ID NO: 33 - RNA, 84 nt, Homo sapien
ggcuguggcuggauucaaguaauccaggauaggcuguuuccaucugugaggccuauucuugauuacuuguuucuggaggcagcu

SEQ ID NO: 34 - RNA, 83 nt, Homo sapien
guggucucagaaucgggguuuugagggcgagaugaguuuauguuuuauccaacuggcccucaaagucccgcuuuuggggucau

SEQ ID NO: 35 - RNA, 85 nt, Homo sapien
ccuuagcagagcuguggagugugacaaugguguuugugucuaaacuaucaaacgccauuaucacacuaaauagcuacugcuaggc

SEQ ID NO: 36 - RNA, 110 nt, Homo sapien
gccgagaccgagugcacagggcucugaccuaugaauugacagccagugcucucgucuccccucuggcugccaauuccauaggucacagguauguucgccucaaugccagc

SEQ ID NO: 37 - RNA, 74 nt, Homo sapien; identical to SEQ ID NO: 2

SEQ ID NO: 38 - RNA, 77 nt, Homo sapien
ggcagugcucuacucaaaaagcugucagucacuuagauuacaugugacugacaccucuuugggugaaggaaggcuca

SEQ ID NO: 39 - RNA, 112 nt, Homo sapien
ccaccccgguccugcucccgccccagcagcacacugugguuuguacggcacuguggccacguccaaaccacacugugguguuagagcgagggugggggaggcaccgccgagg

SEQ ID NO: 40 - RNA, 97 nt, Homo sapien
cagaucucagacaucucggggaucaucaugucacgagauaccagugugcacuugugacagauugauaacugaaaggucugggagccacucaucuuca

SEQ ID NO: 41 - RNA, 87 nt, Homo sapien
ugucccccccggcccagguucugugauacacuccgacucgggcucuggagcagucagugcaugacagaacuugggcccggaaggacc

SEQ ID NO: 42 - RNA, 71 nt, Homo sapien
uguucuaaggugcaucuagugcagauagugaaguagauuagcaucuacugcccuaagugcuccuucuggca

SEQ ID NO: 43 - RNA, 83 nt, Homo sapien
ccuuggaguaaaguagcagcacauaaugguuuguggauuuugaaaaggugcaggccauauugugcugccucaaaaauacaagg

SEQ ID NO: 44 - RNA, 97 nt, Homo sapien
cauuggcaucuauuagguuggugcaaaaguaauugcgguuuuugccauuacuuucaguagcaaaaaucucaauuacuuuugcaccaacuuaauacuu

SEQ ID NO: 45 - RNA, 106 nt, Homo sapien
cccagccuggcacauuaguaggccucaguaaauguuuauuagaugaauaaaugaaugacucaucagcaaacauuuauugugugccugcuaaagugagcuccacagg

SEQ ID NO: 46 - RNA, 129 nt, Homo sapien
gggaugccacauucagccauucagcguacagugccuuucacagggaggugucauuuaugugaacuaaaauauaaauuucaccuuucugagaaggguaauguacagcaugcacugcauaugugguguccc

SEQ ID NO: 47 - RNA, 127 nt, Homo sapien
ggaugccacauucagccauucagugugcagugccuuucacagggaggugucauuuaugugaacuaaaauauaaauuucaccuuucugagaaggguaauguacagcaugcacugcauauguggugucc

SEQ ID NO: 48 - RNA, 82 nt, Homo sapien
ugagcccucggaggacuccauuuguuuugaugauggauucuuaugcuccaucaucgucucaaaugagucuucagaggguucu

SEQ ID NO: 49 - RNA, 75 nt, Homo sapien
gugagggcaugcaggccuggauggggcagcugggaugguccaaaaggguggccucaccagcccuguguucccuag

SEQ ID NO: 50 - RNA, 97 nt, Homo sapien
aaucuaucacugcuuuuuaggauaagcuugacuuuuguucaaauaaaaaugcaaaaggaaaguguauccuaaaaggcaaugacaguuuaauguguuu

SEQ ID NO: 51 - RNA, 84 nt, Homo sapien
uugacuuagcuggguaguggggaacccuuccaugaggaguagaacacuccuuaugcaagauucccuucuaccuggcuggguugg

SEQ ID NO: 52 - RNA, 86 nt, Homo sapien
agcacugcccccggugagucaggguggggcuggcccccugcuucgugcccauccgcgcucugacucucugcccaccugcaggagcu

SEQ ID NO: 53 - RNA, 96 nt, Homo sapien
gcgggcggccccgcggugcauugcuguugcauugcacgugugugaggcgggugcagugccucggcagugcagcccggagccggccccuggcaccac

SEQ ID NO: 54 - RNA, 96 nt, Homo sapien
cucgggaggggcgggagggggguccccggugcucggaucucgagggugcuuauuguucgguccgagccugggucucccucuuccccccaacccccc

SEQ ID NO: 55 - RNA, 96 nt, Homo sapien
ucccuggcgugaggguaugugccuuuggacuacaucguggaagccagcaccaugcaguccaugggcauauacacuugccucaaggccuaugucauc

SEQ ID NO: 56 - RNA, 97 nt, Homo sapien
cucaggugcucuggcugcuuggguuccuggcaugcugauuugugacuuaagauuaaaaucacauugccagggauuaccacgcaaccacgaccuuggc

SEQ ID NO: 57 - RNA, 118 nt, Homo sapien
gcaggugaacuggcaggccaggaagaggaggaagcccuggaggggcuggaggugauggauguuuuccuccgguucucagggcuccaccucuuucgggccguagagccagggcuggugc

SEQ ID NO: 58 - RNA, 72 nt, Homo sapien
agauacuguaaacauccuacacucucagcuguggaaaguaagaaagcugggagaaggcuguuuacucuuucu

SEQ ID NO: 59 - RNA, 110 nt, Homo sapien
ugaguuuugagguugcuucagugaacauucaacgcugucggugaguuuggaauuaaaaucaaaaccaucgaccguugauuguacccuauggcuaaccaucaucuacucca

SEQ ID NO: 60 - RNA, 110 nt, Homo sapien
agaagggcuaucaggccagccuucagaggacuccaaggaacauucaacgcugucggugaguuugggauuugaaaaaaccacugaccguugacuguaccuugggguccuua

SEQ ID NO: 61 - RNA, 110 nt, Homo sapien
ccugugcagagauuauuuuuuaaaaggucacaaucaacauucauugcugucgguggguugaacuguguggacaagcucacugaacaaugaaugcaacuguggccccgcuu

SEQ ID NO: 62 - RNA, 89 nt, Homo sapien
cugauggcugcacucaacauucauugcugucgguggguuugagucugaaucaacucacugaucaaugaaugcaaacugcggaccaaaca

SEQ ID NO: 63 - RNA, 110 nt, Homo sapien
cggaaaauuugccaaggguuugggggaacauucaaccugucggugaguuugggcagcucaggcaaaccaucgaccguugaguggacccugaggccuggaauugccauccu

SEQ ID NO: 64 - RNA, 137 nt, Homo sapien
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 65 - RNA, 88 nt, Homo sapien
caccuuguccucacgguccaguuuucccaggaaucccuuagaugcuaagauggggauuccuggaaauacuguucuugaggucaugguu